(19) United States
(12) Patent Application Publication
(10) Pub. No.: US 2020/0319163 A1
(43) Pub. Date: Oct. 8, 2020

(54) METHODS AND COMPOSITIONS OVERCOMING CANCER CELL IMMUNE RESISTANCE

(71) Applicant: Georgetown University, Washington, DC (US)

(72) Inventor: Louis M. Weiner, Washington, DC (US)

(21) Appl. No.: 16/835,045

(22) Filed: Mar. 30, 2020

Related U.S. Application Data
(60) Provisional application No. 62/825,986, filed on Mar. 29, 2019.

(51) Int. Cl. G01N 33/50; A61K 31/519

(52) U.S. Cl. CPC G01N 33/5011; A61K 31/519

(57) ABSTRACT

Methods and compositions are provided for preventing the loss of cell surface adhesion molecules and thus prevent or delay acquisition of a ADCC resistance phenotype mediated by lower expression of several cell surface molecules that contribute to cell:cell interactions and immune synapse formation including tumor target antigens, MHC Class I/II molecules and cell adhesion proteins. In certain embodiments, the method and composition blocks STAT1 and/or p-STAT1. In another embodiment, the method and composition blocks HATp300 and/or PCAF. In another embodiment the method and composition blocks S100a9/a8. In yet other embodiments, cocktails of combinations of inhibitors of two or more of p-STAT1, S100a8/a9, HAT p300, and PCAF are employed to reduce, reverse or inhibit development of ADCC resistant phenotypes.

### CROSS-REFERENCE TO RELATED APPLICATIONS

This application claims the benefit of U.S. Provisional App. No. 62/825,986, filed Mar. 29, 2019, the entire contents of which are hereby incorporated by reference.

### STATEMENT REGARDING GOVERNMENT INTERESTS

This invention was made with government support under Grant Nos. R01 CA50633, awarded by the National Cancer Institute of the National Institutes of Health. The government has certain rights in the invention.

### FIELD OF THE INVENTION

This invention relates generally to overcoming the resistance of cancer cell to immune mediated cytotoxicity.

### BACKGROUND OF THE INVENTION

Without limiting the scope of the invention, its background is described in connection with previously known mechanisms by which cancer cells are able to evade destruction by the immune system.

Targeted monoclonal antibody therapy is a promising therapeutic strategy for cancer, and antibody-dependent cell-mediated cytotoxicity (ADCC) represents a crucial mechanism underlying these approaches. However, the majority of patients have limited responses to monoclonal antibody therapy due to the development of resistance. Antibody-dependent cell-mediated cytotoxicity (ADCC) was first described as a mechanism of action for monoclonal antibody therapy more than 30 years ago. Most efforts to understand the modulation of ADCC depend upon the incubation of potential effector cells with cytokines or chemokines that modify effector cell function. Relatively little is known about the mechanism by which tumor cells develop resistance to ADCC. Prior studies have examined only a restricted number of candidate genes/proteins (e.g., epidermal growth factor receptor [EGFR] network or receptor tyrosine kinases linked to PD-L1 expression (e.g., JAK1 and JAK2).

### SUMMARY OF THE INVENTION

In one embodiment disclosed herein methods and compositions are provided for preventing the loss of cell surface adhesion molecules and thus prevent or delay acquisition of a ADCC resistance phenotype mediated by lower expression of several cell surface molecules that contribute to cell:cell interactions and immune synapse formation including tumor target antigens, MHC Class I/II molecules and cell adhesion proteins. In one embodiment the method and composition blocks STAT1 and/or p-STAT1. In another embodiment the method and composition blocks HATp300 and/or PCAF. In another embodiment the method and composition blocks S100a9/a8. In certain embodiments, cocktails of combinations of inhibitors of two or more of p-STAT1, S100a8/a9, HAT p300, and PCAF are employed to reduce, reverse or inhibit development of ADCC resistant phenotypes.

In one embodiment, a model is provided wherein a tumor cell of a particular cell type is repeatedly challenged by ADCC to induce a resistant phenotype. Tumor cell types include carcinomas, sarcomas, lymphoma and leukemias, germ cell tumors and blastomas. In certain embodiments the model is employed to identify inhibitors of the development of an ADCC resistant phenotype. In other embodiments, the model is employed to identify agent able to reverse an ADCC resistant phenotype once developed.

### BRIEF DESCRIPTION OF THE DRAWINGS

For a more complete understanding of the present invention, including features and advantages, reference is now made to the detailed description of the invention along with the accompanying figures:

FIG. 1A shows an in vitro NK cell-mediated ADCC model system consisting of an NK-like cell line (NK92-CD16V), an EGFR monoclonal antibody (cetuximab; filled circles), and EGFR-expressing A431 cells. The combination of target, effector, and antibody creates optimal conditions for ADCC. FIG. 1B illustrates a model of derived ADCC resistance according to one embodiment. FIG. 1C shows a time course of A431 cell survival in response to ADCC exposure conditions. FIG. 1D shows specific lysis of 20,000 ADCCR1 cells and 20,000 ADCCS1 cells by NK92-CD16V cells in the presence of cetuximab (1 μg/mL) for 4 hours at a 1:1 E:T ratio. FIG. 1E shows in vitro proliferation of ADCCS1 cells and ADCCR1 cells in the absence of ADCC conditions. FIG. 1F shows growth of subcutaneous tumors derived from ADCCS1 and ADCCR1 cells in Balb/c nude mice. FIG. 1G shows influence of secreted factors by ADCCR1 cells on ADCC sensitivity of ADCCS1 cells.

FIG. 2A shows a representative flow cytometry analysis of EGFR cell-surface expression in ADCCS1 cells and ADCCR1 cells with isotype control expression for ADCCS1 and ADCCR1. FIG. 2B shows EGFR expression in ADCCS1 and ADCCR1 cells measured by mRNA, proteomic, and phosphoproteomic analysis. FIG. 2C shows western blot for EGFR protein expression in ADCCS1 and ADCCR1 cells. FIG. 2D shows representative flow cytometry analysis of HER2 cell-surface expression in ADCCS1 and ADCCR1 cells with isotype control expression for ADCCS1 and ADCCR1. FIG. 2E shows specific lysis of ADCCR1 (target) and ADCCS1 (target) cells by NK92-CD16V (effectors) cells at a 1:1 E:T ratio in the presence of trastuzumab. FIG. 2F shows ADCC-induced specific lysis percentage and corresponding EGFR expression geometric mean by flow cytometry in ADCCS1 cells and in ADCCR1 cells as a function of serial in vitro passaging following the cessation of ADCC exposure.

FIG. 3A shows a heat map of the gene-expression profile of ADCCR1 and ADCCS1 cells using the Illumina HumanHT-12 v4 Expression BeadChip array. FIG. 3B depicts a volcano plot showing that EGFR and HSPB1 showed the most significant loss of gene expression in the ADCCR1 cells, whereas CD74 was the most enriched.). FIG. 3C depicts western blots of JAK1, STAT1, NFκB p65, and p-NFκB p65 in ADCCS1 and ADCCR1 cells. ADCCS1 and ADCCR1 cells post 33 challenges passaged for 3-4 times were used. Densitometry values of expression relative to GAPDH indicated below the blots. FIG. 3D shows a diagram of 300 genes found to be upregulated in ADCCR1 cells compared with ADCCS1 cells by CoGAPS analysis. Overexpressed (red) and underexpressed (green) genes in ADCCR1 compared with ADCCS1 cells. The interferon-induced and histone-associated gene clusters are identified in the bottom right portion of the diagram within hatched boxes.

FIGS. 4A-4C show the effects of pharmacologic modification of histone-associated proteins identified by CoGAPS gene-expression analysis on ADCC sensitivity. FIG. 4A, Western blot of PCAF (KAT2B) in ADCCS1 and ADCCR1 cells. ADCCS1 and ADCCR1 cells post 33 challenges passaged for 3-4 times were used. Densitometry values of expression relative to GAPDH indicated below. FIG. 4B, Specific lysis of ADCCS1 (blue) and ADCCR1 (red) measured by ADCC assay at 4 hours when pretreated with increasing concentrations of histone acetyl transferase inhibitor (HATi) C646 for 2 hours. FIG. 4C, Specific lysis of ADCCS1 (blue) and ADCCR1 (red) measured by ADCC assay at 4 hours when pretreated with increasing concentrations of DNMT (DNMTi), pan-HDAC (pan-HDACi), and histone demethylase inhibitors.

FIGS. 5A-5D depict NK cell activation and conjugation to EGFR+target cells under ADCC conditions. FIG. 5A, Representative dot plots of NK activation measured by flow cytometry analysis using CD107a (APC) and GFP+NK92-CD16V cells at a 1:1 E:T for 2 hours as described in Materials and Methods. ADCCS1 (top row) and ADCCR1 cells (bottom row) were incubated with NK92-CD16V cells in the absence (middle) or presence of cetuximab (1 μg/mL; right) for 2 hours. FIG. 5B, ELISA measuring IFNγ levels in the media of ADCCS1 (blue bars) and ADCCR1 (red bars) 4 hours after exposure to ADCC conditions (cetuximab [1 μg/mL] plus NK92-CD16V cells) at E:T ratios of 0-4:1 and NK92-CD16V cells in the absence of cetuximab. FIG. 5C, Western blot analysis of granzyme B and perforin protein expression in target cells 2 hours after exposure to T (media control), Ab (cetuximab only at 1 μg/mL), NK (NK92-CD16V cells only at 1:1 E:T), and ADCC (NK92-CD16V cells at 1:1 E:T plus 1 μg/mL cetuximab). FIG. 5D, Percentage of NK cells conjugated to target was measured by a multiwell conjugation assay as described in Materials and Methods.

FIGS. 6A-6D depict cell-surface screens of ADCCS1 and ADCCR1 cells. FIG. 6A, Dot plot comparing geometric means of ADCCS1 and ADCCR1 cell-surface molecule expression measured by the BD Lyoplate assay. FIG. 6B, Geometric means of molecules with the highest differential expression (box in A) in ADCCS1 (blue) compared with ADCCR1 (red) cells. FIG. 6C, Representative histograms of selected cell-surface molecules with reduced cell-surface expression on ADCCR1 cells based on lower protein expression. FIG. 6D, Representative histograms of selected cell-surface molecules with reduced cell-surface expression based on reduced transport to cell surface. FIG. 6E presents the effect of blockade of CD54 on ADCC.

FIG. 7A shows morphological features of ADCCS1 cells. FIG. 7B shows morphological features of ADCCR1 cells.

FIG. 8A shows morphological features of A431 cells. FIG. 8B shows morphological features of ADCCR1 cells after 26 challenges.

FIG. 9A shows a heat map comparing significantly different (p<0.05) protein expression in ADCCS1 and ADCCR1 cells. Replicates represent technical replicates of ADCCS1 and ADCCR1. IPA was used to generate the heat map and calculate Z-score which represents the number of standard deviations from the mean of a normal distribution of activity edges. FIG. 9B shows a heat map of comparing significantly different (p<0.05) protein phosphorylation in ADCCS1 and ADCCR1 cells. Replicates represent technical replicates of ADCCS1 and ADCCR1. IPA was used to generate the heatmap and calculate Z-score which represents the number of standard deviations from the mean of a normal distribution of activity edges.

FIG. 10 shows differentially phosphorylated proteins (p<0.05) in ADCCS1 and ADCCR1 cells, respectively.

FIG. 11A shows HSP27 expression in ADCCS1 and ADCCR1 cells reflected by mRNA, proteomic and phosphoproteomic analysis. Z-score (the standard scaling function in R), a centered and scaled vector that allows comparison between vectors with different orders of magnitude, was used to compare expression between ADCCS1 and ADCCR1. FIG. 11B shows western blots of HSP27 in ADCCS1 and ADCCR1 cells. Densitometry value of expression relative to GAPDH indicated below. FIG. 11C shows western blots of HSP27 in control ADCCS1 and ADCCR1 cells (−) and ADCCS1 and ADCCR1 cells overexpressing HSP27 (OE). Densitometry value of expression relative to GAPDH indicated below. FIG. 11 D shows specific lysis of control ADCCS1 and ADCCR1 cells (solid bars) and ADCCS1 and ADCCR1 cells overexpressing HSP27 (checkered bars) as measured by ADCC assay. FIG. 11E shows CD74 expression in ADCCS1 and ADCCR1 cells reflected by mRNA and proteomic analysis. Z-score (the standard scaling function in R), a centered and scaled vector that allows comparison between vectors with different orders of magnitude, was used to compare expression between ADCCS1 and ADCCR1. FIG. 11F shows flow cytometry analysis of CD74 cell surface and total protein expression in ADCCR1 cells. Light gray histogram: negative control. Dark gray histogram: expression of total CD74 protein in permeabilized ADCCR1 cells. Open histogram: cell surface expression of CD74 in ADCCR1 cells.

FIG. 12A shows Ingenuity Pathway Analysis generated top canonical pathways employed by genes enriched in ADCCR1 cells by CoGAPS Analysis. FIG. 12B shows Ingenuity Pathway Analysis generated upstream regulators of genes enriched ADCCR1 cells by CoGAPS analysis.

FIG. 12C shows gGene expression intensity measured by whole genome Illumina bead array of IFN pathway genes in ADCCR1 and ADCCS1 cells.

FIG. 13 shows the effect of ICAM-1 blockade on ADCC sensitivity.

FIG. 14 shows immune checkpoint cell surface expression in ADCCS1 and ADCCR1 cells.

FIGS. 15A-15E show characterization data on a second ADCC resistant cell line. FIG. 15A shows specific lysis of A431 cells as measured by ADCC assay as a function of consecutive ADCC challenges. FIG. 15B, Flow cytometry analysis of EGFR cell surface expression in ADCCR2 at challenge 48 and cells from the four challenge treatment conditions (untreated control, Ab treatment only, NK treatment only, and ADCC conditions) at challenge 49. FIG. 15C: NK activation measured by flow cytometry analysis using CD107a (APC) and GFP+NK92-CD16V cells as described in Materials and Methods. ADCCS2 (top row) and ADCCR2 cells (bottom row) were incubated with NK92-CD16V cells in the absence (middle panels) or in the presence of 1 μg/ml of cetuximab (right panel) for 2 hours. FIG. 15D: Percentage of NK cells conjugated to target was measured by multiwell conjugation assay as described in Materials and Methods. ADCCS2 (light blue) and ADCCR2 (orange) were incubated with NK92-CD16V cells in the absence (NK, checkered bar) or in the presence of 1 μg/ml of cetuximab (ADCC, solid bar) for 2 hours. FIG. 15E: Geometric means of ICAM-1 expression as measured by flow cytometric analysis in ADCCS1 (blue), ADCCR1 (red), ADCCS2 (light blue), and ADCCR2 (orange).

FIGS. 16A-16F show the relative expression of a panel of cell surface molecules in ADCCS1 and ADCCR1.

FIGS. 17A-17C show expression of the markers γH2AX, p53, p-p53, STAT1, p-STAT1, CD74 and PCAF over a number of successive ADCC challenges.

FIG. 18A-18D show expansion of pre-existing resistant clones. 10× scRNAseq analysis was conducted and identified a ADCCS1 cell with an ADCCR1 resistance signature. FIG. 18A shows the results of CoGAPS analysis of bulk gene expression profiling. FIG. 18B applies ProjectR to transfer the resistance signature learned in bulk data. FIG. 18C shows UMAP analysis clusters suggesting incomplete penetrance of the resistant phenotype, while FIG. 18D reveals that similar trends are observed in pseudotime analysis.

FIG. 19 depicts the results of single cell RNAseq analysis of 1) ADCCS1, 2) ADCCS1 24 hours following ADCC challenge and 3) ADCCR1 cells for CD74, EGFR, KAT2B (PCAF), STAT1, and p53

FIG. 20 presents a pathway of resistance development.

FIG. 21A shows the role of T-cell immunity in a pancreatic cancer cell model where MT3-2D pancreatic cancer cells are introduced into immunocompetent (WT) C57Bl/6 mice versus SCID C57/BL mice and T-cell depleted WT mice.

FIG. 21B shows the pathways significantly enriched in WT vs SCID and the reciprocal.

FIG. 22 shows a table of which STAT1 and myeloid genes were selectively expressed by tumor cells in response to immune attack.

FIG. 23 presents a preliminary model on the RNAseq data.

FIG. 24 presents data showing that STAT1 overexpression is associated with poorer overall survival in human PDAC.

FIGS. 25A and 25B present data showing that S100a8/a9 is selectively increased in WT tumors by proteomic analysis, is not expressed by tumor cells, but is clearly induced by T cell immunity. This molecule is controlled in part by STAT1, binds to RAGE and activates MDSC.

FIG. 26 presents data showing that neutrophilic myeloid derived suppressor cells (G-MDSC) selectively accumulate in WT mouse tumors.

FIG. 27 presents the new pathways uncovered leading to immune and immunotherapy resistance and defines new targets for combination therapy.

FIG. 28 presents the results of a single cell RNAseg analysis of PARP1, MUS81, STAT3, TP53BP1, STAT1, BRCA1, CDKN1A, XRCC3, TP53, RAD17, ATM, PRKDC, MDM2, EFGR, and SFN with activation signature in ADCC resistance.

FIG. 29 presents the results of a cytotoxicity analysis of ADCCS1 and ADCCR1 after ruxolitinib exposure.

FIG. 30A-D present data showing that ADCC resistance is associated with reduced cell surface expression of multiple proteins. FIG. 30A shows CD99 and MUC1 expression. FIG. 30B shows CD54 expression in ADCCS1 and ADCCR1 cells. FIG. 30C shows CD54 protein expression. FIG. 30D shows CD73 protein expression.

FIG. 31A-C present data showing that ADCC resistance is accompanied by the loss of CD54. FIG. 31A shows CD54 expression in ADCC resistant cells. FIG. 31B shows that a CD54 blockade reduces ADCC in ADCC sensitive cells. FIG. 31C shows intracellular sequestration of CD54 is associated with Golgi complexes.

FIG. 32 presents the results of ICAM1 re-expression on ADCC sensitivity.

FIG. 33A-C present data showing the ADCC resistance mechanism reproduced in multiple cell lines. FIG. 33A shows the ADCC resistance mechanism in A431 cell lines. FIG. 33B shows the ADCC resistance mechanism in SKOV3 cell lines. FIG. 33C shows the ADCC resistance mechanism in FaDu cell lines.

FIG. 34 shows the results of LEGENDscreen of surface molecules of ADCC sensitive and ADCC resistant cells of A431, SKOV3, and FaDu cell lines.

FIG. 35 shows the results of immunoblotting the expression of CD49f and GAPDH in ADCC sensitive and ADCC resistant cells of the A431, SKOV3, and FaDu cell lines.

FIG. 36 shows the results of a flow cytometry analysis of ADCCS1 and ADCCR1 cells for expression of EGFR and the cetuximab-binding epitope o EGFR.

FIG. 37 shows the results of immunofluorescent imaging for cetuximab in ADCCS1 and ADCCR1 cells.

FIG. 38 shows the results of immunofluorescent imaging for cetuximab and trastuzumab in ADCC sensitive and ADCC resistant cells in the FaDu and SKOV3 cell lines.

FIG. 39A-B shows the results of flow cytometric and immunofluorescence analysis, demonstrating that ADCC-resistant cells exhibit loss of binding of directly-labeled antibody to target antigens. FIG. 39A shows flow cytometry based binding of commonly used flow antibodies to target cells. Blue—Sensitive Cells; Salmon—Resistant Cells; Mauve—Negative Control. FIG. 39B shows directly-labeled antibodies binding to ADCC-resistant cells: Cetuximab (A431, FaDu) or trastuzumab (SK-OV-3), conjugated to Dylight 550 (1 mg/ml). Blue: DNA. 40X.

FIG. 40A-D show the effects of ATMi, ATM, siP53, and RUX (ruxolitinib) on cell lysis in ADCCS1 and ADCCR1 cells. FIG. 40A shows cell lysis rates for ATMi and ATM. FIG. 40B shows cell lysis rates for 200 nM siP53. FIG. 40C shows cell lysis rates for 100 nM siP53. FIG. 40D shows cell lysis rates for 10 nM ruxolitinib.

### DETAILED DESCRIPTION OF THE INVENTION

The present inventor appreciated that a model of ADCC would provide a system for uncovering immune-resistance mechanisms and developed and characterized such a model. In one embodiment of a model system provided herein, epidermal growth factor receptor (EGFR⁺) A431 tumor cells were continuously exposed to Killer cell immunoglobulin-like receptor (KIR)-deficient NK92-CD16V effector cells together with the anti-EGFR monoclonal cetuximab. This persistent ADCC exposure yielded ADCC-resistant cells (ADCCR1) that, compared with control ADCC-sensitive cells (ADCCS1), exhibited reduced EGFR expression, overexpression of histone- and interferon-related genes, and a failure to activate NK cells, without evidence of epithelial-to-mesenchymal transition. These properties were found to gradually reversed following withdrawal of ADCC selection pressure. The development of ADCC resistance was associated with lower expression of multiple cell-surface molecules that contribute to cell-cell interactions and immune synapse formation. Classic immune checkpoints did not modulate ADCC in this unique model system of immune resistance. As disclosed herein, it was determined that the induction of ADCC resistance involves genetic and epigenetic changes that lead to a general loss of target cell adhesion properties that are required for the establishment of an immune synapse, killer cell activation, and target cell cytotoxicity.

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within 5%, within 1%, and in certain aspects within 0.5%.

The present disclosure represents a comprehensive analysis of tumor cell-based resistance mechanisms to ADCC, which serves as a model for the induction of resistance to continuous immune attack. Hence, some of the mechanisms that emerge may be anticipated to occur in response to other mechanisms of immune attack, such as cytotoxic T-cell attack through the formation of immune synapses. To ensure consistency, NK92-CD16V cell line was used as an effector, which many have used to explore various facets of ADCC. This model system permitted the inventor to explore mechanisms of ADCC resistance unrelated to known KIR molecule-related regulatory NK cell mechanisms, as has been previously demonstrated. Resistance mechanisms identified in the current study, thus, may be relevant to other forms of immune attack, such as T-cell receptor-mediated cytolysis. Future studies will address these possibilities.

2 distinct ADCC-resistant cell lines have been isolated from A431 cells, termed ADCCR1 and ADCCR2. In comparison with parental ADCC-sensitive A431 cells, ADCCR1 is characterized by reduced cell-surface expression of EGFR and other molecules associated with cell adhesion and immune synapse formation, reduced expression of HSPB1 (a known chaperone of EGFR), and increased expression of CD74 (a known MHC II chaperone and regulator of antigen presentation). ADCCR1 cells had a distinct transcriptional profile characterized by upregulation of genes associated with interferon response and histone function. The ADCCR1-resistance phenotype was partially reversed by inhibition of the histone acetyltransferase p300. ADCCR1 cells proliferated more slowly than A431 cells, and ADCCR1 tumors also grew more slowly in nude mouse xenografts. The ADCCR2 cell line, which was induced using a similar ADCC selection pressure strategy, did not exhibit reduced cell-surface EGFR expression.

In this model system, it has been demonstrated that a loss of numerous cell-surface molecules is associated with adhesion and immune synapse formation. NK cell activation is a dynamic process mediated by multiple factors, many of which promote adhesion. ADCCR1 and ADCCR2 cells exhibited significant loss of ICAM-1, a known LFA-1 ligand that mediates the tight adhesion between target cells and cytotoxic lymphocytes required for cytotoxic activity of T cells and NK cells. This was evident by the inhibitory effect of blocking LFA-1/ICAM-1 interactions on ADCC and NK cell natural cytotoxicity that many have observed. NFκB p65 activity has been inversely associated with ICAM-1 expression and consequently ICAM-1/LFA-1 binding and NK cytotoxicity. ADCCR1 cells overexpressed CD74 in the cytoplasm but not on the cell surface. This finding was of some interest, as the cytoplasmic tail of CD74 regulates NFκB activity.

In this model system, ADCC resistance was not associated with PD-L1 expression, in contrast to the findings described by others implicating IFNγ in the upregulation of PD-L1 and resistance. The findings indicated that, in contrast to establishment of immune blockade, ADCCR1 and ADCCR2 cells achieved resistance to immune attack by lowering cell-surface expression of molecules known to mediate cell adhesion and formation of the immune synapse in order to prevent immune cell conjugation. The distinct EGFR expression in ADCCR1 and ADCCR2 cells demonstrated that altered levels of EGFR expression are independent of changes in cell adhesion molecule expression and sensitivity to ADCC. Ultimately, reduced NK cell conjugation, activation, and degranulation are likely the result of a combination of factors, including but not limited to loss of cell adhesion receptors and/or EGFR. The ADCC resistance phenotype described here is believed to represent an adaptive mechanism to shield cells from immune attack.

The reversibility of the resistance phenotype, coupled with a histone-related gene signature in the ADCCR1 cells, suggested this was an epigenetic phenomenon, linked to interferon response genes and CD74 upregulation, the induction of NFκB p65, and then modulation of cell-surface receptor expression to reduce the conjugation of effector cells. Although ADCC resistance is likely the result of multiple cellular changes, the modification of the immune synapse function is of particular interest. It is speculated that similar phenotypes could be induced by powerful immune selection through ADCC or similar mechanisms that involve the formation of immune synapses. This hypothesis is supported by the findings that the resistance phenotype reverted back to the ADCC-sensitive phenotype after continuous culture in non-ADCC conditions.

These findings address important questions related to the induction of cellular resistance to ADCC and possibly other immune therapies. It has long been assumed that therapy-imposed selection pressures would induce genetic or epigenetic changes to permit targeted cells to escape immune control. Epigenetic modifications are established tumorigenic mechanisms. Earlier studies have linked anticancer drug resistance to epigenetic modification leading to transcriptional silencing of genes necessary for drug activation. However, its role in cancer immunopathology and immunotherapy is poorly understood. The IFNγ pathway has been linked to primary, adaptive, and acquired resistance to checkpoint blockade therapy. Prolonged exposure to IFNγ can lead to immune escape due to cell desensitization and immune editing. The immune system can be hindered by epigenetic changes within the target cell, which prevent the recruitment or activation of effector cells. A study has demonstrated that epigenetic suppression of TH1 chemokines suppresses cell trafficking to the tumor microenvironment. Multiple studies have shown that epigenetic modification by HDAC inhibitors alone or in combination with DNMT inhibitors can enhance immunotherapy.

It should be noted that tumor cell resistance to T-cell attack has been known to involve defective antigen presentation, depriving killer cells of their targets. Similarly, target antigen modulation is a known mechanism of resistance to monoclonal antibody therapy. Here, it has been shown that the induction of ADCC resistance could lead to the more general loss of target cell adhesion properties required for the establishment of an immune synapse, killer cell activation, and target cell cytotoxicity. In contrast to models of cellular cytotoxicity resistance that invoke the establishment of immune checkpoints, this work demonstrates that target cells can evade conjugation by rendering the cells invisible to the cytotoxic apparatus.

The following examples are included for the sake of completeness of disclosure and to illustrate the methods of making the compositions and composites of the present invention as well as to present certain characteristics of the compositions. In no way are these examples intended to limit the scope or teaching of this disclosure.

### Example 1

### Materials and Methods

Cell Lines and Cell Culture:

The A431 cell line was obtained from the Georgetown Lombardi Tissue Culture Shared Resource in 2010 and 2014, and its origin was verified by DNA fingerprinting by short tandem repeat analysis prior to utilization. The A-431 cell line is also obtainable from the ATCC as CRL-1555 and was derived from a human epidermoid carcinoma. ADCCS1 and ADCCR1 were derived from cells obtained in 2010 and have been used during 2010-2018. Tissue culture growth conditions for this cell line were high-glucose Dulbecco's modified Eagle medium (DMEM; HyClone) supplemented with 10% fetal bovine serum (FBS; Omega Scientific) and 2 mmol/L (1×) 1-glutamine (Gibco). NK92-CD16V cells that express GFP due to transduction with pBMN-IRES-EGFP were kindly provided by Kerry S. Campbell from Fox Chase Cancer Center (Philadelphia, Pa.). They were cultured in MEMa-modification (HyClone) supplemented with 10% FBS, 10% horse serum, 1 mmol/L sodium pyruvate, and 1× nonessential amino acids (Gibco), as well as 0.1 mmol/L β-mercaptoethanol (Sigma) as described (Weiner L M, et al. Monoclonal antibodies: versatile platforms for cancer immunotherapy. Nat Rev Immunol 2010; 10:317-27.). NK92-CD16V cells were maintained in suspension and passaged every 2-3 days by resuspending the cells in NK media (described above) at a concentration of 0.2×106 cells/mL and stimulated with 1% v/v of IL2 supernatant derived from J558L cells (Binyamin L, et al. Blocking NK cell inhibitory self-recognition promotes antibody-dependent cellular cytotoxicity in a model of anti-lymphoma therapy. J Immunol 2008; 180:6392-401). All cell lines were maintained at 37° C. in 5% CO₂ and tested negative for Mycoplasma. Cell counts were estimated by hemocytometer, and viable cells identified by trypan blue (Invitrogen) exclusion.

Inhibitors and Treatment Antibodies:

Inhibitors of histone acetyltransferase C646 (cat. no. S7152), DNA methyltransferase azacytidine (cat. no. S1782), histone demethylase GSK J4 HCL (cat. no. S7070), and HDAC panobinostat (cat. no. S1030) were purchased from Selleck Chemicals. Inhibitors were solubilized in DMSO at 20 μmol/L. Vehicle treatment (DMSO) was used at the highest equivalent v/v used in inhibitor treatments. Cells/well (10,000) of both ADCCS1 and ADCCR1 were plated overnight in 96-well, clear-bottom white plates (Corning, cat. no. 3903), then treated in the presence of the inhibitors at 0.01 to 10 μmol/L concentrations for 2 hours prior to ADCC assay. Cetuximab (Bristol-Myers Squibb) and trastuzumab (Genentech) were purchased from the MedStar Georgetown University Hospital Pharmacy.

Flow Cytometry:

A431 cells were cultured for 3 to 6 passages and then were dissociated using 0.25% trypsin, resuspended in DMEM plus 10% FBS and 1% 1-glutamine. Cells (0.5×10⁶ to 1×10⁶) were aliquoted into Eppendorf tubes, spun at 5,000 rpm for 1 minute at 4° C., washed twice with HBSS (Fisher Scientific; cat. no. SH3058801), and resuspended in 100 μL of FACS buffer (PBS plus 1% BSA). All antibodies used are labeled antibodies, and no blocking step was performed. Labeled antibodies were then added at the manufacturer's recommended concentrations and incubated at 4° C. for 30 minutes, with vortexing at 15 minutes. For intracellular staining, cells were resuspended in 50 μL of BD perm/wash (cat. no. 554723) for 20 minutes before proceeding to staining with antibody at 4° C. for 30 minutes. Cells were then washed with FACS buffer twice and resuspended in FACS buffer or fixative (1% PFA in PBS). Flow antibodies were purchased from BioLegend: EGFR (cat. no. 352904), CD74 (cat. no. 326807), CD54/ICAM (cat. no. 322713), CD142 (cat. no. 365205), CD73 (cat. no. 344021), ITGB4/CD104 (cat. no. 343903), ALCAM/CD166 (cat. no. 343903), CD95/Fas (cat. no. 305611), CD138, (cat. no. 352307), and APC-labeled IgG1 isotype control (cat. no. 400121). CD107a (cat. no. 641581), CD44 (cat. no. 559942), HER2 (cat. no. 340879), and PD-L1 (cat. no. 557929) were purchased from BD Biosciences. PE-labeled IgG1 isotype control was purchased from eBioscience (cat. no. 12-4714-81). Samples were run in the Georgetown Lombardi Comprehensive Cancer Center Flow Cytometry and Cell Sorting Shared Resource using BD LSRFortessa. Analyses were performed using FlowJo (v10.4.1).

Derivation of ADCC Resistance: Initial Derivation of ADCC Resistance.

A431 cells were seeded overnight in 6-well plates (Greiner Bio-One; cat. no. 657160) at 150,000 cells per well. The following day, 6 different treatment groups were added for the initial ADCC challenge: (i) vehicle (media); (ii) cetuximab (0.01 or 1 μg/mL); (iii) 500,000 NK92-CD16V cells, cetuximab (0.01 μg/mL) plus 500,000 NK92-CD16V cells (low ADCC), or cetuximab (1 μg/mL) and 500,000 NK92-CD16V cells (high ADCC). Adding 500,000 NK92-CD16V cells under these culture conditions equates to ˜2:1 effector-to-target (E:T) ratio at the time of treatment addition. Three or 4 days later, all wells were aspirated of treatments, washed, and the remaining adherent cells were collected by trypsinization. Viable cell density for each treatment was assessed by trypan blue exclusion. Identical conditions were used for each subsequent ADCC challenge. Over 6 months, 34 consecutive, subsequent challenges were conducted. Viable cell density was used as a surrogate to assess for resistance in the treatment groups. After every fifth treatment cycle (Ch5, Ch10, Ch15, etc.), cells from each treatment were also expanded for one passage and cryopreserved.

Rederivation of ADCC Resistance:

A431 cells were seeded overnight in 5 T75 flasks (Greiner Bio-One; cat. no. 658175) at 500,000 cells per flask. The following day, the flasks were divided into 4 treatment groups: untreated (media only), cetuximab (1 μg/mL), 1×10⁶ NK cells (1:1 E:T), and ADCC (1 μg/mL cetuximab plus 1:1 E:T). Each of the control groups contained 1 flask, and the ADCC group was distributed into 2 flasks to allow for sufficient cell numbers when pooled to replate and expand for cryopreservation, Western blot, flow cytometry, and ADCC assays. Treatments were applied for 72 hours, and then the flasks were aspirated, the cells were washed, and the remaining adherent cells were collected by trypsinization. Viable cell density for each treatment was assessed by trypan blue exclusion. Forty-nine additional challenges were conducted. Resistance in ADCC treatment groups was assessed by morphology, cell proliferation rate, and ADCC assay.

ADCC Assay:

ADCC assays were performed in 96-well, clear-bottom white plates (Corning; cat. no. 3903) using the Cytotox-Glo Cytotoxicity assay (Promega; cat. no. G291). ADCC assays were preformed using A431/ADCCS1/ADCCR1 as target cells and NK92-CD16V cells as the effector cells. Target cells are plated at 10,000 cells/well overnight (A431 cells double overnight). Specific lysis was assessed at 4 hours after exposure to NK92-CD16V cells (20,000 cells/well) at 1:1 E:T ratio in the presence or absence of cetuximab (1 μg/mL) or trastuzumab (5 μg/mL) as described (Murray J C, et al. c-Abl modulates tumor cell sensitivity to antibody-dependent cellular cytotoxicity. Cancer Immunol Res 2014; 2:1186-98).)

For assessment of specific lysis after blocking ICAM-1, cells were plated in medium containing the blocking antibody (10 m/mL; BioLegend; cat. no. 322703).

Western Blot:

Cells were lysed in boiling buffer with EDTA (Boston BioProducts) supplemented with 1× protease and 1% phosphatase inhibitor prepared following the manufacturer's protocols (Sigma-Aldrich; cat. no. 11697498001 and P5726). Cleared lysate concentrations were obtained by a DC Protein Assay (Bio-Rad). Lysates 30 to 40 μg were run on SDS-PAGE gels and transferred to nitrocellulose membranes (GE Healthcare). Western blots were conducted using the Abcam antibodies to EGFR (cat. no. 52892) and perforin (cat. no. ab180773), and Cell Signaling Technology antibodies to GAPDH (cat. no. 5174), JAK1 (cat. no. 3332), STAT1 (cat. no. 14994), p-STAT1 Y701 (cat. no. 9167), PCAF/KA2B (cat. no. 3378), granzyme B (cat. no. 4275), NFκB p65 (cat. no. 82420), p-NFκB P65 (cat. no. 3031S), and HSPB1 (cat. no. 2402S). Goat anti-rabbit or donkey anti-mouse IgG HRP-conjugated secondary antibodies (GE Healthcare) were used with chemiluminescence substrates (Pierce). Densitometry was measured using ImageJ (v1.48).

NK Cell Activation Assay:

CD107a was used as a marker of NK cell degranulation and activation. ADCCS1 and ADCCR1 cells were seeded overnight in 6-well plates at 500,000 and 700,000 cells, respectively. The effect of ADCCS1 and ADCCR1 cells on the activation of NK cells in the presence and absence of cetuximab was examined. CD107a expression on unexposed NK cells, ADCCS1, and ADCCR1 cells was also measured to ensure no autofluorescence and background. NK cells (1×10⁶) and cetuximab for final 1 μg/mL concentration were added to each well. The exposure time was 2 hours, after which cells were collected and stained as described in the flow cytometry methods. Samples were run in the Georgetown Lombardi Comprehensive Cancer Center Flow Cytometry and Cell Sorting Shared Resource using BD LSRFortessa. Analyses were performed using FlowJo (v10.4.1).

NK Cell Conjugation Assay:

NK conjugation was assessed using a multiwell conjugation assay. Target cells (ADCCS1 or ADCCR1) were plated at a density of 10,000 cells per well on 96-well clear-bottom black plates (Greiner, 655090) in FluoroBrite DMEM (Gibco) supplemented with 10% FBS and incubated overnight at 37° C., 5% CO₂. NK92-CD16V cells at a density of 8×10⁵ cells/mL in Dulbecco's PBS were labeled with 5 μmol/L carboxyfluorescein diacetate (Molecular Probes) for 20 minutes at 37° C., 5% CO₂. The labeled NK92-CD16V cells were spun at 1500 rpm for 5 minutes and resuspended in NK medium (described above) and incubated for an additional 10 minutes at 37° C., 5% CO₂. The labeled NK92-CD16V cells were spun again at 1,500 rpm for 5 minutes and resuspended in the FluoroBrite DMEM to 8×10⁵ cells/mL. NK cells (25 μL representing ˜1:1 E:T) were added in sextuplets to target cells. Then, either 25 μL of medium or cetuximab (1 μg/mL) was added to target cells. As background, 50 μL of medium alone was added to a row of target cells. The plate was incubated for 2 hours, and then initial fluorescence was read using a PerkinElmer's Envision 2104 Multilabel Reader set to 492/517 nm excitation/emission. Wells were emptied of nonadherent NK cells, washed twice with 200 μL of FluoroBrite DMEM, refilled with 150 μL FluoroBrite DMEM, and ending fluorescence was measured. The percentage of NK cells in conjugate was calculated as [(fluorescenceend−fluorescencebackground)/(fluorescenceinitial−fluorescencebackground)]×100. The mean of all replicates for each target cell line was then determined and SEM calculated.

Cell-Surface Screen:

The BD Lyoplate Human Cell-Surface Marker Screening Panel (BD Biosciences; 560747) contains purified monoclonal antibodies to 242 cell-surface markers. ADCCS1 and ADCCR1 cell lines were compared. Each cell line was screened twice. The cells were dissociated from flasks using BD Accutase (cat. no. 561527) and resuspended in BD Pharmingen stain buffer (FBS; cat. no. 554656) at 5×106 cells/mL. Cells 100 μL/well (5×105 cells) were then dispensed into three 96-well round-bottom plates (BD Falcon; 353910). The assay was conducted according to the manufacturer's instructions. Samples were run in the Georgetown Lombardi Comprehensive Cancer Center Flow Cytometry and Cell Sorting Shared Resource using BD LSRFortessa. The flow cytometry analysis was done using FlowJo (v10.4.1).

Viability and Proliferation Assays:

ADCCS1 and ADCCR1 cells were plated at 1,000 cells/well and 2,000 cells/well in 96-well plates (Fisher Scientific; cat. no. 720089), respectively. Seven plates were prepared for each cell line to measure proliferation across 7 days without treatment or with effector cell exposure. CellTiter-Blue (Promega) assays were conducted in 96-well format per manufacturer's instructions on one plate per cell line for 7 days to measure in vitro proliferation of ADCCS1 and ADCCR1. Prism GraphPad 5 was used to conduct two-tailed t tests and P value.

ELISA Assays:

Human IFNγ ELISA MAX Deluxe Kit (BioLegend, 430104) was used to measure IFNγ in the media 4 hours after ADCC exposure. ADCCS1 and ADCCR1 cells were plated in 96-well clear-bottom plates (Corning, 3300) at 10,000 cells/well and incubated in culture conditions overnight at 37° C. in 5% CO₂. The control wells were then exposed to either media, cetuximab (1 μg/mL), or NK92-CD16V cells at the indicated E:T ratios in the absence of antibody. The ADCC wells all were incubated with cetuximab (1 μg/mL) and NK92-CD16V cells, reflecting E:T ratios of 0:1, 1:1, 2:1, and 4:1 by adding 0; 20,000; 40,000; and 80,000 NK cells, respectively, to the wells. After 4-hour incubation, the plates were spun down at 1,000×g for 5 minutes, and the supernatant was collected and transferred into a fresh round-bottom plate. IFNγ detection in supernatants was done using the ELISA MAX Deluxe Kit (BioLegend; cat. no. 430105) according to the manufacturer's instructions.

In Vivo Tumor Growth:

Cohorts of ten 6- to 8-week-old female BALB/c nude mice were injected subcutaneously (s.c.) in the right flanks with 1×106 cells of ADCCS1 or 2×106 ADCCR1 cells suspended in 100 μL PBS. Tumor size was monitored twice weekly and measured using a caliper, and the volume was calculated using the following formula: Volume=(½)×length×width. Animals were euthanized when tumors reached 2 cm in the largest diameter or exhibited undue suffering. All animal experiments were carried out with Georgetown University Institutional Animal Care and Use Committee approval.

RNA Isolation and Gene-Expression Analysis:

Six pairs (12 total samples) of serially passaged vehicle-treated ADCCS1 cells and ADCCR1 cells from challenges 30 to 35 were passaged twice without treatments and collected by trypsinization. RNA was isolated using the PureLink RNA Mini Kit (Ambion). RNA quality was assessed for quality by Bioanalyzer (Agilent) for an RNA Integrity Number (RIN)>6. The direct hybridization assay method (as per the manufacturer's instructions) was used to generate biotin-labeled cRNA from 100 ng of RNA, which was then hybridized to the HumanHT-12 v4 Expression BeadChip, washed, and scanned per the manufacturer's instructions (Illumina). All data were obtained from a single BeadChip. Data have been submitted to the Gene Expression Omnibus (GEO) repository, GEO accession number GSE114545.

Data were preprocessed with log 2 variance stabilization and quantile normalization using the R/Bioconductor package lumi and subset to detected probes. Differential expression analysis was performed with the R/Bioconductor package LIMMA, using unpaired, empirical Bayes moderated t tests to compare sensitive and resistant cells. Probes with false discovery rate (FDR)-adjusted P values below 0.01 were called statistically significant.

Coordinated Gene Activity in Pattern Sets (CoGAPS) analysis and PatternMarker statistics were performed for time-course analysis. Probes with less than 1 log fold change between any 2 samples were filtered from analysis. Mean and standard deviation for probes annotated to the same gene were computed. Standard deviations were assigned to be the maximum of 10% of the mean gene-expression value or standard deviation computed across all probes. These gene-level data summaries were input to CoGAPS, and the algorithm was run for a range of 2 to 8 patterns, with 5 found to be optimal fit based upon ClutrFree analysis. Three of the 5 patterns inferred changes in transcription across the passages and 2 stable changes between sensitive and resistant cells across all passage numbers, the latter of which were selected for further analysis. PatternMarker genes for the pattern upregulated in resistant cells were input to STRING (Mering von C, et al.. STRING: a database of predicted functional associations between proteins. Nucleic Acids Res 2003; 31:258-61; version 6.2) to generate networks. Gene-level expression values were z-scored across all samples and visualized in the STRING network using the R package network.

Sample Preparation for Proteomics and Phosphoproteomics:

Cell pellets from ADCCS1 and ADCCR1 cells were resuspended in lysis buffer containing 50 mmol/L Tris HCl, pH 7.5, 150 mmol/L NaCl, 1% Triton X-100, 5 mmol/L EDTA, 1× Protease Inhibitor Cocktail (Roche; cat. no. 04693132001)) and 1× Phosphatase Inhibitor Cocktail (Sigma; cat. no. P5726). The suspension was sonicated using a probe-tip ultrasonic processor (Vibra Cell; with the AMPL setting of 30%) 2 times for 10 seconds and spun down at 12,000×g for 15 minutes. The supernatant was collected, with proteins extracted by methanol/chloroform precipitation. The precipitated proteins were then dissolved in 8 M urea and 50 mmol/L triethylammonium bicarbonate, pH 8, with the protein concentration determined by the BCA assay (Thermo Fisher, cat. no. 23225). Equal amounts (50 μg for proteomics and 300 μg for phosphoproteomics) of proteins from each sample were reduced with 10 mmol/L DTT for 30 minutes at 37° C. and alkylated with 30 mmol/L iodoacetamide for 30 minutes at room temperature in the dark, followed by quenching with 10 mmol/L DTT for another 30 minutes. After decreasing the urea concentration with 50 mmol/L triethylammonium bicarbonate to 1 M, sequencing-grade trypsin (Promega) was added and incubated overnight at 37° C. After acidification with trifluoroacetic acid (final: 2%), tryptic digests were desalted with C18 spin columns (Nest Group) and dried with a SpeedVac. Each sample was then labeled with one isotopic reagent in a 6-plex iTRAQ labeling kit (Sciex) according to the manufacturer instructions. Differentially labeled peptides were then pooled and dried by vacuum centrifugation. Dried peptide mixtures were then fractionated with an Agilent 1260 Infinity HPLC system by using a C18 column (3.5 μm 2.1×100 mm XTerra MS; for proteomics) or another C18 column (5 μm 4.6×250 mm)(Bridge; for phosphoproteomics) with a 60-minute gradient of buffer A (20 mmol/L ammonium formate in H2O, pH 10) and buffer B (20 mmol/L ammonium formate in ACN, pH10). All the fractions were collected (1 fraction for every 1 minute) and combined into 12 fractions with a concatenation method (14). Phosphoproteomic samples were processed with one more step: after being dried with a SpeedVac, phosphopeptides in each fraction were enriched with a Titansphere Phos-Tio Kit (GL Sciences), according to manufacturer instructions.

NanoUPLC-MS/MS:

Dried peptides and phosphopeptides from each fraction were dissolved into 20 μL of 0.1% formic acid. Each sample (1 μL for proteomics and 10 μL for phosphoproteomics) was loaded onto a C18 Trap column (Waters Acquity UPLC Symmetry C18 NanoAcquity 10 K 2G V/M, 100 A, 5 μm, 180 μm×20 mm) at 15 μL/minute for 4 minutes. Peptides were then separated with an analytical column (Waters Acquity UPLC M-Class, peptide BEH C18 column, 300 A, 1.7 μm, 75 μm×150 mm), which was temperature controlled at 40° C. The flow rate was set at 400 nL/minute. A 90-minute gradient of buffer A (2% ACN, 0.1% formic acid) and buffer B (0.1% formic acid in ACN) was used for separation: 1% buffer B at 0 minute, 5% buffer B at 1 minute, 40% buffer B at 80 minutes, 99% buffer B at 85 minutes, 99% buffer B at 90 minutes. The gradient went back to 1% buffer B in 10 minutes, with the column equilibrated with 1% buffer B for 20 minutes. Data were acquired using an ion spray voltage of 2.3 kV, GS1 5 psi, GS2 0, CUR 30 psi and an interface heater temperature of 150° C. Mass spectra were recorded with Analyst TF 1.7 (AB SCIEX) in the information-dependent acquisition (IDA) mode. Each cycle consisted of a full scan (m/z 400-1,600) and 50 IDAs (m/z 100-1,800) in the high-sensitivity mode with a 2+ to 5+ charge state. Rolling collision energy was used, with iTRAQ reagent collision energy adjustment on.

Proteomic and Phosphoproteomic Data Analysis:

Data files were submitted for simultaneous searches using Protein Pilot version 5.0 software (Sciex) utilizing the Paragon and Progroup algorithms and the integrated FDR analysis function. MS/MS data were searched against the NCBI Homo Sapiens Proteome (UP000005640) of the UniProt-Sprot database containing 20,316 entries (Filtered by reviewed and downloaded on Jun. 2, 2015). For proteomics, “Trypsin” was selected as the enzyme, “Carbamido-methylation” was set as a fixed modification on cysteine. Variable peptide modifications included methionine (M) oxidation and iTRAQ labeling of the N-terminal lysine (K) and tyrosine (Y). For phosphoproteomics, search parameters were set as follows: sample type [iTRAQ-8plex], cys alkylation (Iodoacetamide), digestion (Trypsin), instrument (TripleTOF 5600), special factors (phosphorylation emphasis), species (Homo Sapiens), ID Focus (Biological modifications), database (uniprot_sprot.fasta), search effort (Thorough), FDR analysis (Yes), and user-modified parameter files (No). The proteins were inferred based on the ProGroup™ algorithm associated with the ProteinPilot software. Peptides were defined as redundant if they had identical cleavage site(s), amino acid sequence, and modification. All peptides were filtered with confidence to 5% FDR, with the confidence of phosphorylation sites such as phospho-serine (p-Ser), phospho-threonine (p-Thr), and phospho-tyrosine (p-Tyr) automatically calculated. Quantitative phosphopeptide selection criteria are as follows: (i) The phosphopeptides without quantitative information were discarded. (ii) The phosphor peptides that were annotated with “autodiscordant peptide-type” and “autoshared MS/MS” were excluded. For both data sets, the detected protein threshold in the software was set to the value that corresponded to 1% FDR. Automatic normalization of quantitative data (bias correction) was performed to correct any experimental or systematic bias.

Statistical Analysis:

Statistical analysis done in in vitro cell proliferation, in vivo tumor growth, specific lysis, target:NK cells conjugation cell viability was two-tailed t tests conducted using prism GraphPad 5. Gene-expression analysis was conducted via the R/Bioconductor package lumi, and data time-course analysis using CoGAPS analysis and PatternMarker statistics. Proteomic and phosphor proteomic analysis was conducted using the Paragon and Progroup algorithms and the integrated FDR analysis function. Measures of mRNA expression, proteomic and phosphoproteomic peptide counts were normalized by mean-centered scaling across sample groups (Z-score) using R to provide comparable distributions between assay types.

### Example 2

### Deriving Resistance to ADCC

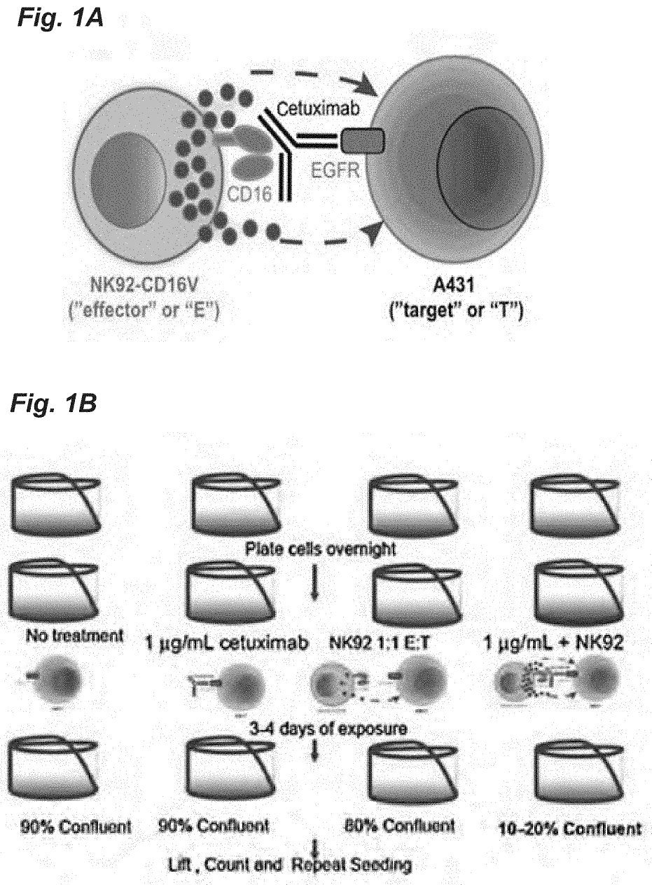

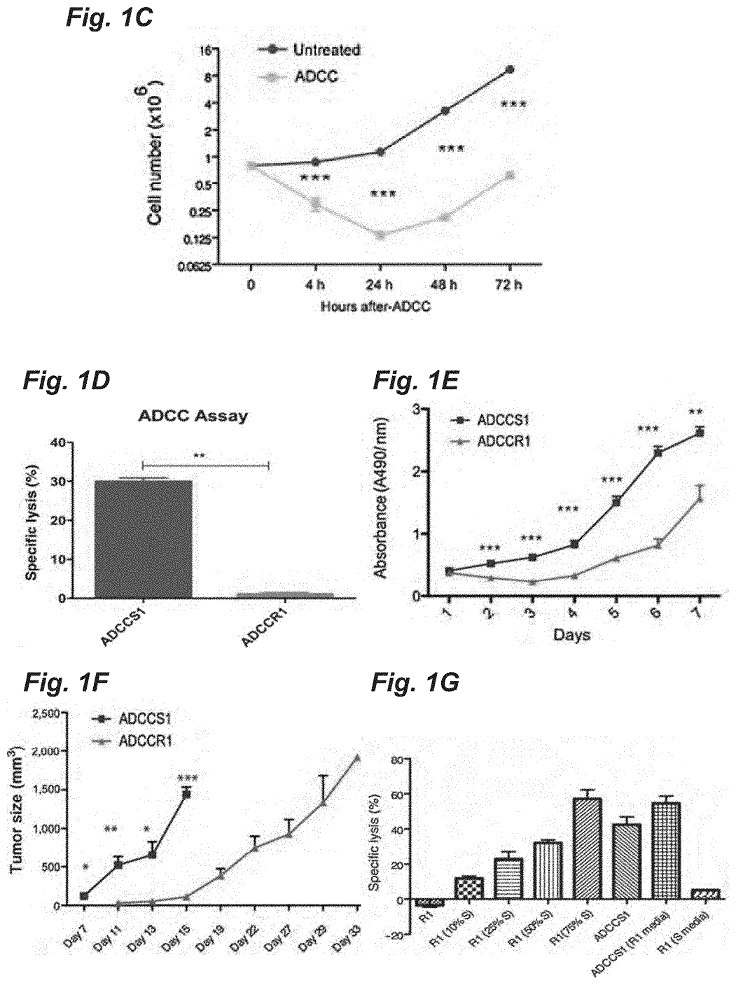

Previously, it had been shown that A431 cells are sensitive to cetuximab-mediated ADCC, using a model system consisting of EGFR-overexpressing A431 cells, NK92-CD16V, and cetuximab (FIG. 1A). (Murray J C, et al. c-Abl modulates tumor cell sensitivity to antibody-dependent cellular cytotoxicity. Cancer Immunol Res 2014; 2:1186-98) FIG. 1A shows An in vitro NK cell-mediated ADCC model system consisting of an NK-like cell line (NK92-CD16V), an EGFR monoclonal antibody (cetuximab; red-filled circles), and EGFR-expressing A431 cells. The combination of target, effector, and antibody creates optimal conditions for ADCC. In order to explore mechanisms of resistance to ADCC and develop a model of ADCC resistance, A431 cells were continuously exposed in vitro to ADCC conditions for 30 to 50 challenges, consisting of the addition of fresh NK cells and cetuximab every 3 days following the removal of exhausted media and nonadherent cells. FIG. 1B depicts a schematic of the workflow of the 4 conditions of continuous exposure. Untreated control, cetuximab (1 μg/mL)-treated control, and NK cell-mediated ADCC in the absence and presence of cetuximab (1 μg/mL).

FIG. 1C shows a time course of A431 cell survival in response to ADCC exposure conditions. A431 cells were seeded and exposed to ADCC conditions for the indicated times, as described in Materials and Methods. ***, P<0.001 by two-tailed t test across all time points as indicated on the graph. Error bars, SEM. FIG. 1D shows specific lysis of 20,000 ADCCR1 cells and 20,000 ADCCS1 cells by NK92-CD16V cells in the presence of cetuximab (1 μg/mL) for 4 hours at a 1:1 E:T ratio. **, P<0.01 by two-tailed t test. Error bars, SEM. FIG. 1E shows in vitro proliferation of ADCCS1 cells and ADCCR1 cells in the absence of ADCC conditions. ***, P<0.001; **, P<0.01 by two-tailed t test for days 2-6 and day 7, respectively. Error bars, SEM. FIG. 1F shows growth of subcutaneous tumors derived from ADCCS1 and ADCCR1 cells in Balb/c nude mice. N=10 in each group. P value calculated by two-tailed t test as indicated on the graph. *, P<0.05; **, P<0.01; ***, P<0.001. Error bars, SEM. FIG. 1G shows influence of secreted factors by ADCCR1 cells on ADCC sensitivity of ADCCS1 cells. Bar graph, ADCCR1 (R1) cells compared with mixed ADCCR1/ADCCS1 (S) cells at indicated percentages. Error bars, SEM.

A431 cell survival in response to ADCC conditions (FIG. 1C) showed the most target cell death (90%) at 24 hours. After 72 hours of ADCC exposure, more than a 90% difference in cell survival between ADCC-treated cells and untreated cells (0.6×10⁶ and 9.3×10⁶ cells, respectively) was still observed. A431 cell numbers had recovered to only 25% of their pretreatment baseline at 48 hours after challenge but were almost fully recovered at 72 hours, demonstrating that 72-hour cycles of ADCC conditions permitted sufficient target cell killing and recovery of residual viable A431 cells to generate conditions permissive for the emergence of ADCC-resistant cells.

After 34 consecutive ADCC challenges, the surviving A431 cells (designated ADCCR1) demonstrated slower proliferation, morphologic changes, and an increased number of cells surviving the ADCC challenge. ADCC sensitivity was assessed and quantified by measuring specific lysis in ADCCR1 cells compared with contemporaneously cultured but untreated A431 cells (ADCCS1). There was a significant difference between ADCC-induced specific lysis in ADCCR1 and ADCCS1 cells (P<0.01 by two-tailed t test; FIG. 1D).

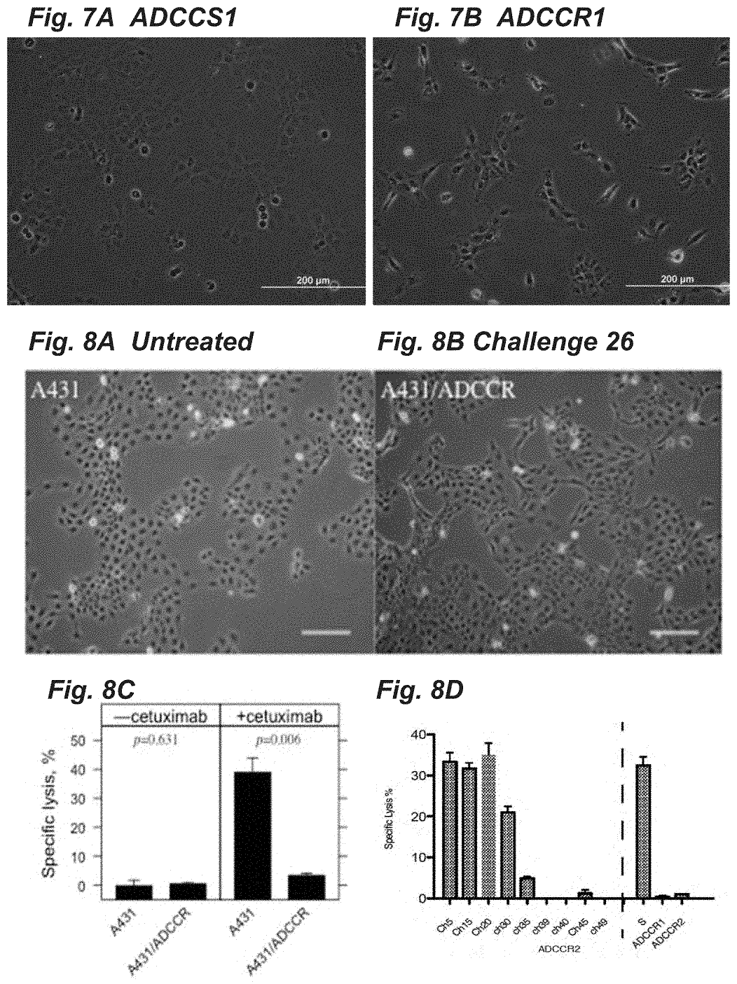

In comparison with ADCCS1 cells, ADCCR1 morphology was elongated with a “spindle-like” appearance reminiscent of fibroblasts, with apparent contrast at cell margins. ADCCR1 cells displayed less distinct colony or clonal organization, with a tendency for reduced cell-cell contact (FIG. 7A versus FIG. 7B and FIG. 8A versus FIG. 8B). Both in vitro proliferation (FIG. 1E) and in vivo subcutaneous xenograft growth in nude mice (FIG. 1F) with ADCCR1 tumors was significantly slower compared with ADCCS1 tumors. ADCCR1 cell proliferation was reduced by 50% (***, P<0.001 by two-tailed t test). Mice bearing ADCCS1 and ADCCR1 tumors had median survivals of 15 and 33 days, respectively.

The possibility was considered that ADCCR1 cells secrete factors that mediate ADCC resistance, and addressed this by admixing ADCCS1 and ADCCR1 cells at varying ratios, and also reciprocally substituting supernatants from ADCCS1 cells with media from ADCCR1 cells (FIG. 1G). Specific lysis correlated with the proportion of ADCCS1 cells added to ADCCR1 (P=0.003, R2=0.904), whereas the media exchanges had no effects on cytotoxicity.

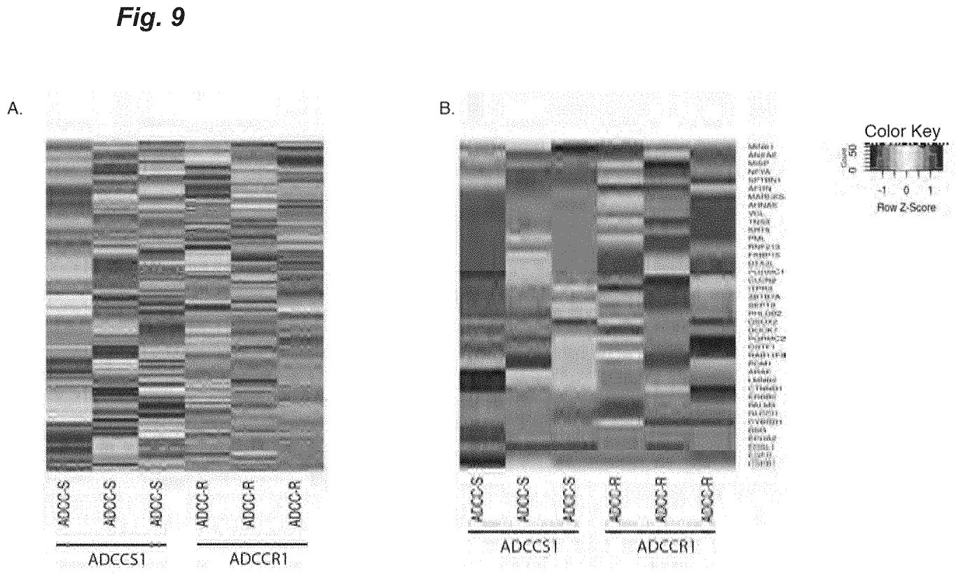

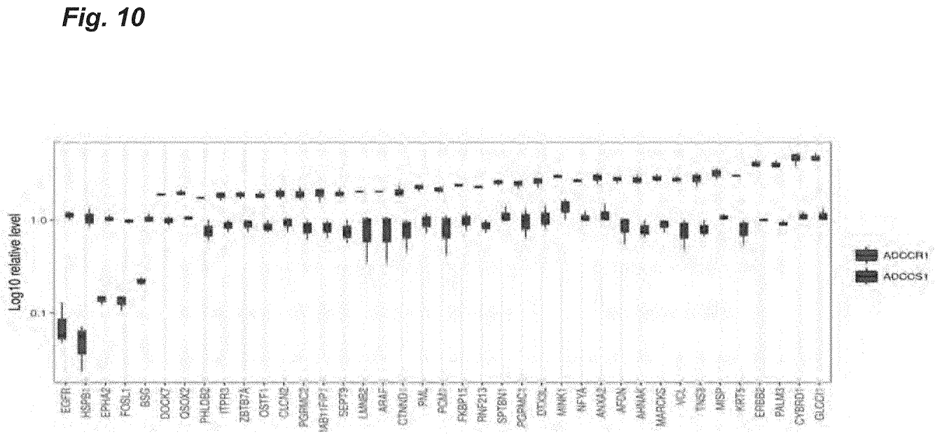

ADCCR1 and ADCCS1 cells possessed significantly different phosphoproteomic and proteomic profiles (FIGS. 9A and 9B). Among the phosphorylated proteins with statistically significantly altered phosphorylation in ADCCR1 versus ADCCS1 cells, a general tendency toward hyperphosphorylation of proteins in the ADCCR1 cells was seen (FIG. 9B). Only 5 proteins were selectively hypophosphorylated in the ADCCR1 cells (FIG. 10). The protein, phosphoprotein, and mRNA of selected proteins in ADCCR1 cells compared with ADCCS1 cells showed a similar pattern across the data sets.

Relation of EGFR Expression to ADCC Resistance:

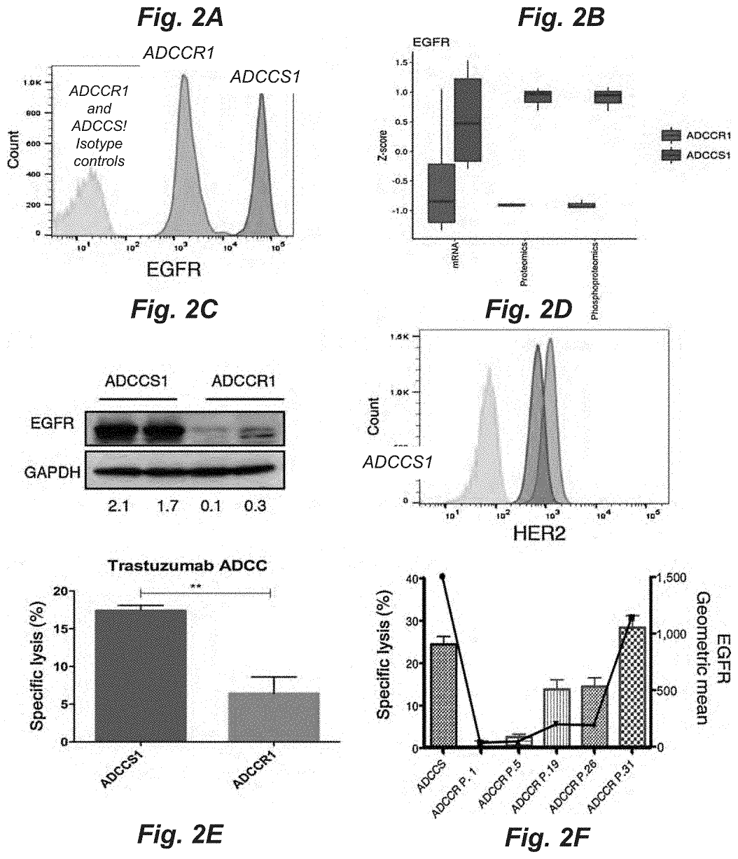

EGFR is the target of cetuximab. Therefore, the role of EGFR in the ADCCR1 cells was investigated to better understand the EGFR association with the ADCC resistance phenotype. EGFR was significantly reduced on the cell surface of ADCCR1 cells compared with ADCCS1 cells (FIG. 2A). EGFR protein had concordantly reduced gene expression in the ADCCR1 cells (FIG. 2B). Reduced EGFR protein expression was also found by proteomic analysis and Western blot, and reduced EGFR phosphorylation was demonstrated by phosphoproteomic analysis (FIGS. 2B and C).

EGFR and HER2 expression in ADCCS1 and ADCCR1 cells. FIG. 2A shows a representative flow cytometry analysis of EGFR cell-surface expression in ADCCS1 cells and ADCCR1 cells with isotype control expression for ADCCS1 and ADCCR1. FIG. 2B shows EGFR expression in ADCCS1 and ADCCR1 cells measured by mRNA, proteomic, and phosphoproteomic analysis. Measures of mRNA expression as well as proteomic and phosphoproteomic peptide counts were normalized by mean-centered scaling across sample groups (Z-score) to provide comparable distributions between assay types. Analysis was done on ADCCS1 and ADCCR1 cells post 33 challenges passaged for 3-4 times. FIG. 2C shows western blot for EGFR protein expression in ADCCS1 and ADCCR1 cells. ADCCS1 and ADCCR1 cells post 33 challenges passaged for 3-4 times were used. Densitometry values of expression relative to GAPDH indicated below the blot. FIG. 2D shows representative flow cytometry analysis of HER2 cell-surface expression in ADCCS1 (blue) and ADCCR1 (red) cells with isotype control expression for ADCCS1 (light blue) and ADCCR1 (light red). FIG. 2E shows specific lysis of ADCCR1 (target) and ADCCS1 (target) cells by NK92-CD16V (effectors) cells at a 1:1 E:T ratio in the presence of trastuzumab (5 μg/mL) for 4 hours. **, P<0.01 by two-tailed t test.). FIG. 2F shows ADCC-induced specific lysis percentage (bars) and corresponding EGFR expression geometric mean by flow cytometry (solid line) in ADCCS1 cells and in ADCCR1 cells as a function of serial in vitro passaging (P, passage number) following the cessation of ADCC exposure.

Next, it was assessed whether the loss of EGFR was responsible for the ADCC-resistant phenotype exhibited by ADCCR1 cells. It has previously demonstrated that EGFR knockdown in parental A431 cells results in a moderate reduction of sensitivity to ADCC. Although the EGFR surface expression, measured by flow cytometry, in the cells with EGFR knockdown was similar to what was observed in ADCCR1 cells, it did not exhibit the complete ADCC resistance displayed by ADCCR1 cells. This indicated that although loss of EGFR contributed to ADCC resistance in ADCCR1 cells, it was not the sole mediator of resistance. Next, the ADCC sensitivity of ADCCR1 cells was examined using a different antibody target. ADCCR1 and ADCCS1 cells express similar levels of HER2 (FIG. 2D). However, ADCCR1 cells displayed resistance to ADCC mediated by trastuzumab (FIG. 2E). This suggested that additional ADCC-resistance mechanisms, beyond EGFR loss, mediate the ADCCR1 phenotype.

ADCC resistance and the EGFR-loss phenotype were not durable in the absence of continued ADCC selection pressure. When ADCCR1 cells were cultured in the absence of cetuximab and NK92-CD16V cells, the expression of EGFR slowly returned to that of wild-type A431 cells over 31 passages (approximately 3 months; FIG. 2F). The restoration of ADCC sensitivity had some correlation with EGFR recovery, and ADCC sensitivity returned rapidly, even with minimal increases in EGFR surface expression.

Overexpression of Interferon- and Histone-Associated Genes in ADCCR1 Cells:

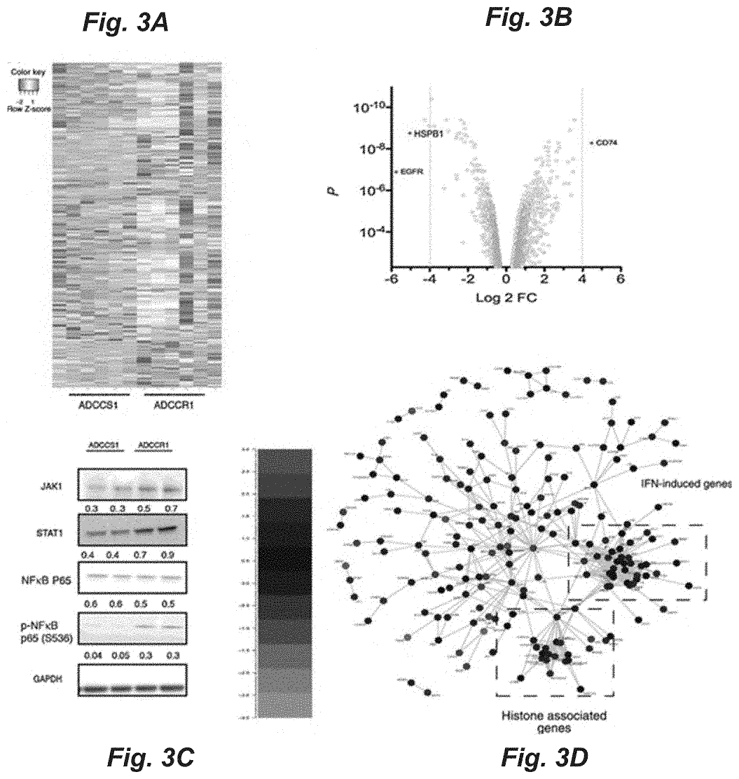

To investigate the difference between ADCC-resistant and -sensitive cells, the gene-expression profile of ADCCR1 and ADCCS1 cells was examined using the Illumina HumanHT-12 v4 Expression BeadChip array. FIG. 3A shows a heat map of gene expression assessed by whole-genome Illumina bead arrays in ADCCS1 and ADCCR1 cells. Differential gene-expression analysis was conducted for genes possessing at least 2-fold changes and an adjusted FDR of P<0.01. The heat map is based on hierarchical clustering of both samples (columns) and probes (rows) and contains 388 total probes for 334 unique genes. Reduced (blue) and increased (red) gene expression is shown based on z-score assessment across each probe (row). The cell lines showed distinct transcriptional profiles (FIG. 3A). As shown in the volcano plot in FIG. 3B of differential gene expression in ADCCR1 compared with ADCCS1 cells, differential gene-expression analysis was conducted for genes possessing an adjusted FDR of P<0.01. The dotted line (vertical) indicates the P value threshold 4 and −4 Log 2 FC indicating significantly upregulated and downregulated genes, respectively (red). EGFR and HSPB1 showed the most significant loss of gene expression in the ADCCR1 cells, whereas CD74 was the most enriched (FIG. 3B). FIG. 3C shows western blots of JAK1, STAT1, NFκB p65, and p-NFκB p65 in ADCCS1 and ADCCR1 cells. ADCCS1 and ADCCR1 cells post 33 challenges passaged for 3-4 times were used. Densitometry values of expression relative to GAPDH indicated below the blots. FIG. 3D provides a diagram of 300 genes found to be upregulated in ADCCR1 cells compared with ADCCS1 cells by CoGAPS analysis. Overexpressed (red) and underexpressed (green) genes in ADCCR1 were compared with ADCCS1 cells. The interferon-induced and histone-associated gene clusters are identified in the bottom right portion of the diagram within hatched boxes. When prosurvival molecules known to associate with CD74 were examined, a selective activation of RELA (NFκB p65) in ADCCR1 cells was found (FIG. 3C).

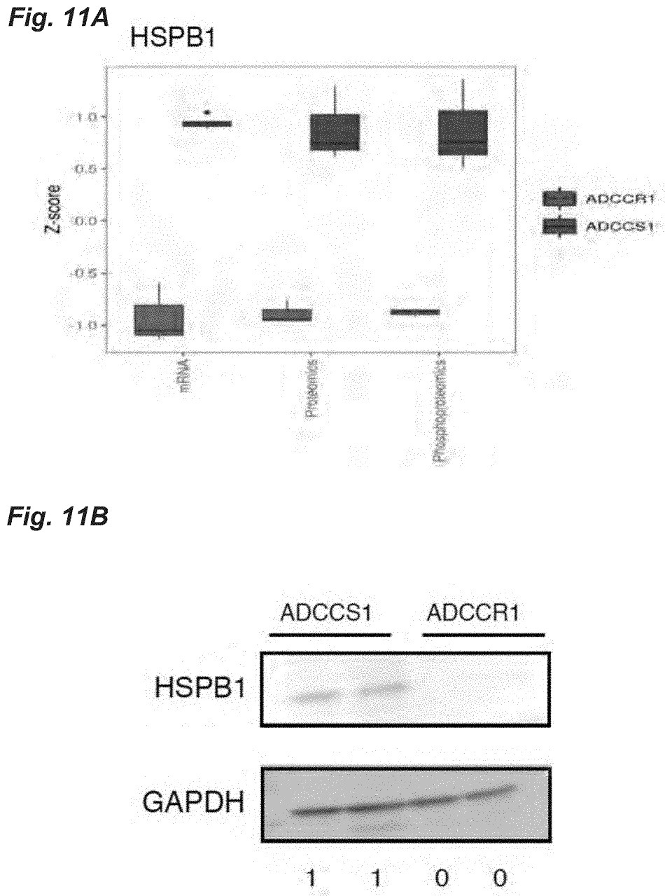

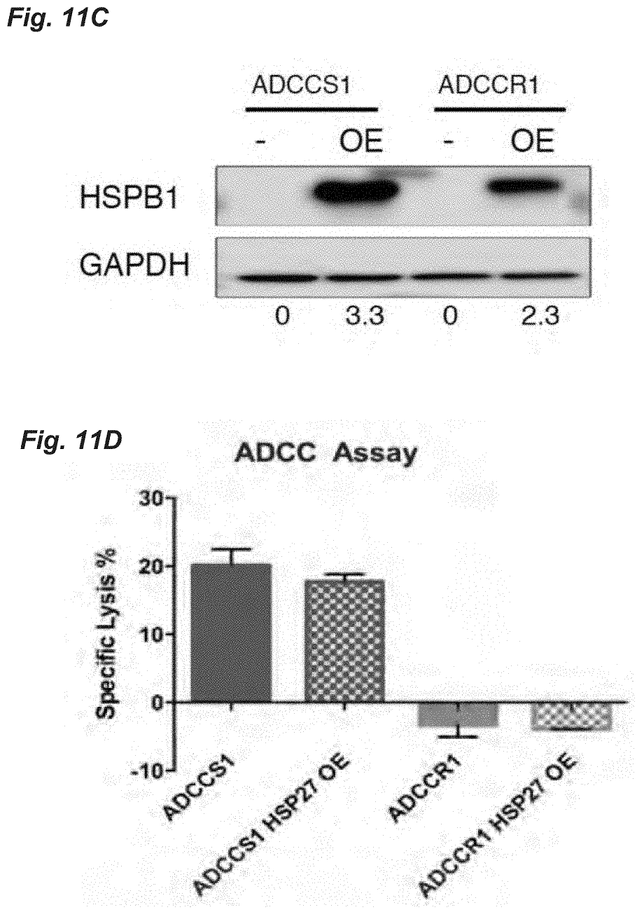

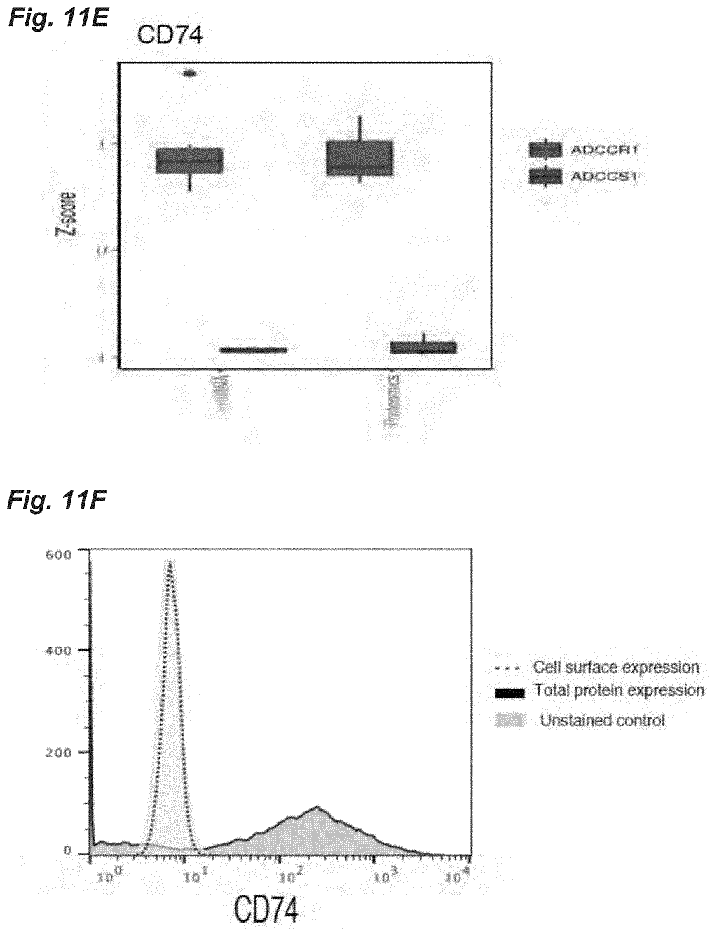

Although HSPB1 loss was found consistently across data sets and validated by Western blot (FIGS. 11A and 11B), overexpression of HSPB1 in ADCCR1 cells did not resensitize ADCCR1 cells to ADCC. Elevation of CD74 in ADCCR1 cells compared with ADCCS1 cells was observed in the proteomic analysis, in addition to the gene-expression analysis (Supplementary Fig. S3E). Total CD74 protein in the cell was significantly higher. However, CD74 was not present on the cell surface (Supplementary Fig. S3F). The cytosolic intracellular domain of CD74 is known to regulate the transcription of cell survival genes (17).

FIG. 11C shows western blots of HSP27 in control ADCCS1 and ADCCR1 cells (−) and ADCCS1 and ADCCR1 cells overexpressing HSP27 (OE). Densitometry value of expression relative to GAPDH indicated below. HSP27 plasmid was purchased from addgene (#63102) and purified using OriGene PowerPrep HP Plasmid MidiPrep (NP #100006). ADCCS1 and ADCCR1 cells were plated in a 6 well plate overnight followed by reverse transfection with 2 μg of purified DNA using Lipofectamine 3000 Transfection Kit (Invitrogen, L3000-008) following standard protocol. Cells were harvested for analysis after 24 hours.

Analysis of ADCCS1 and ADCCR1 cells from challenges 30 to 35 was performed with the CoGAPS algorithm, using the time-course analysis pipeline from Stein-O'Brien and colleagues. The PatternMarker statistic for CoGAPS identified 300 genes with consistent upregulation and 450 genes with consistent downregulation in ADCCR1 cells compared with ADCCS1 cells across all challenges. The 300 genes upregulated in ADCCR1 cells contained clusters of interferon-associated and histone-associated genes (FIG. 3D). No observed upregulation in gene expression of any known marker for epithelial-to-mesenchymal transition was seen.

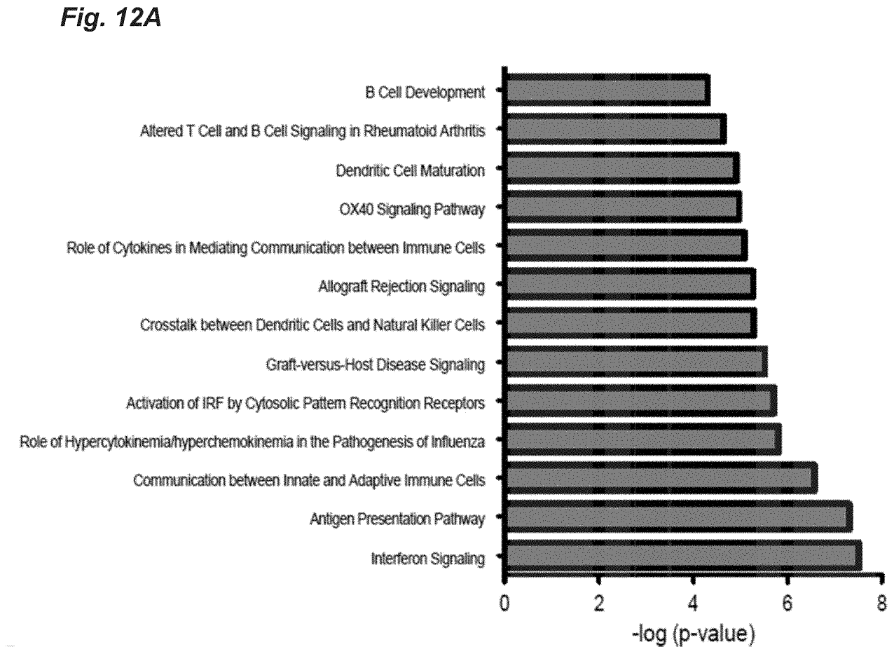

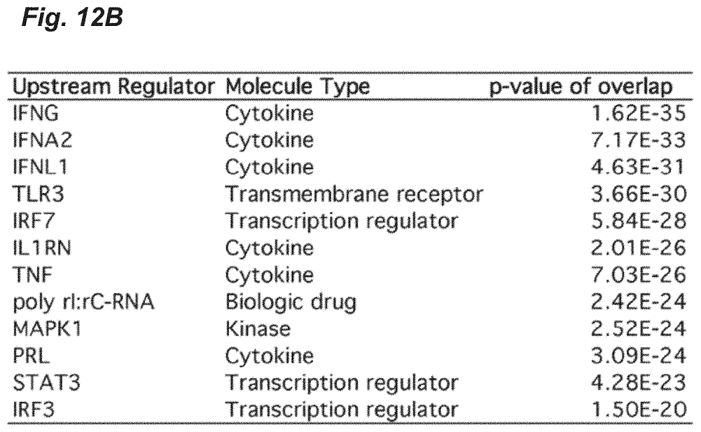

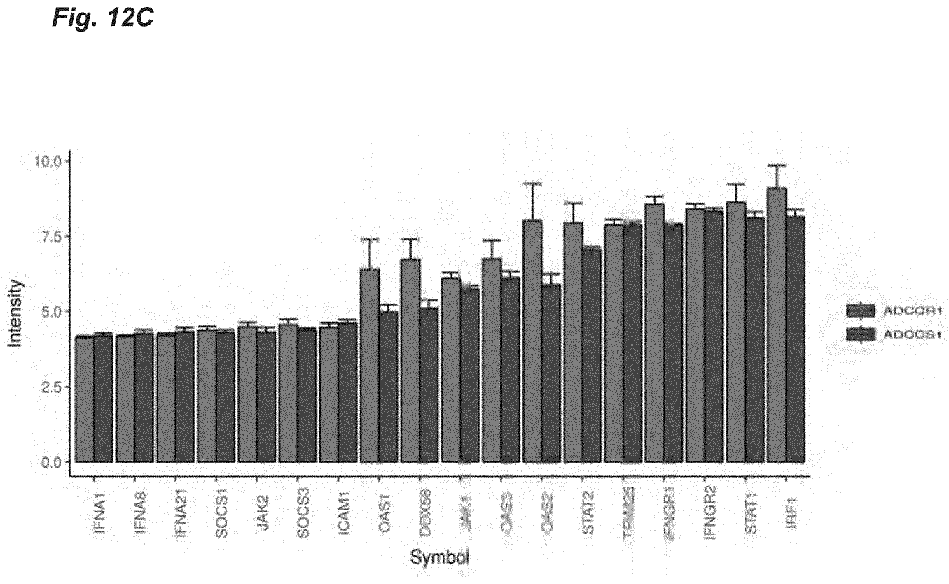

Ingenuity Pathway Analysis was used to analyze the expression pattern of genes upregulated in ADCCR1 cells. Interferon signaling, antigen presentation, and communication between innate and adaptive immune cells were the top canonical pathways identified (FIG. 12A), and IFNγ was found to be a top upstream regulator of these cells with a P value of overlap 1.62×10⁻³⁵ (FIG. 12B). Although IFNγ itself was not significantly overexpressed in these cells, proteins downstream of IFNγ were overexpressed in ADCCR1 compared with ADCCS1 cells (JAK1 and STAT1), further supporting activation of IFN signaling (FIG. 3C).

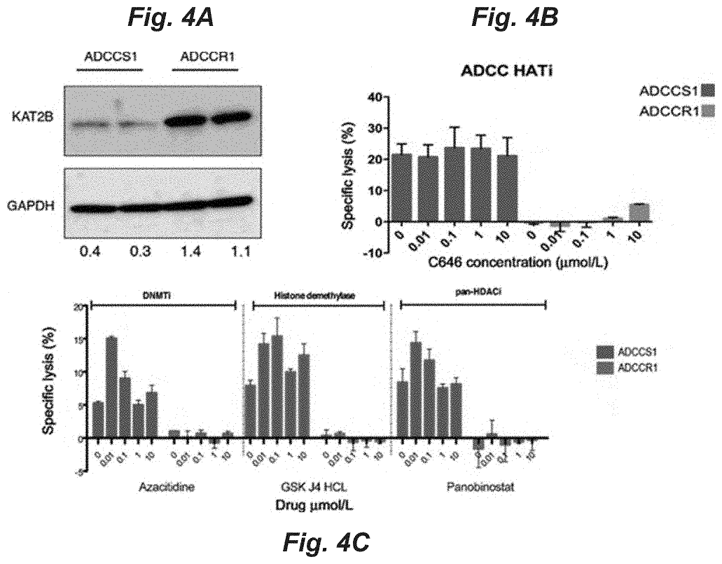

Upregulated histone-associated gene expression (Table 1) pointed to a possible epigenetic mechanism driving ADCC resistance. KAT2B, a p300-associated histone acetyltransferase found within this histone cluster, was relatively overexpressed in ADCCR1 cells compared with ADCCS1 cells (FIG. 4A). ADCC resistance was partially reversed by inhibition of p300 using the histone acetyltransferase inhibitor C646 (FIG. 4B). No resensitization to ADCC in ADCCR1 cells was seen when using pan-HDAC, histone demethylase, or DNMT inhibitors (FIG. 4C).

TABLE 1
Histone and Interferon Induced Genes Upregulated in ADCCR1

| Histone Associated genes | Interferon Associated genes |
|---|---|
| KAT2B | IFI27, IFI6 |
| KDM6A | IFI44, IFI44L |
| HIST1H2AC | IFIH1 |
| HIST1H2BD | IFIT1, IFIT2, IFIT3 |
| HIST1H2BJ | IFNB1 |
| HIST1H2BK | DDX60 |
| HIST1H3G | DHX58 |
| HIST1H3H | HERC6, HERC5 |
| HIST2H2AA3 | UBA7 |
| HIST2H2AA4 | OVOL1 |
| HIST2H2AC | DDX58 |
| HIST2H2BE | IRF6, IRF9 |
| HIST2H4A | ISG15, ISG20 |
| HIST2H4B | HLA-B |
| HIST3H2A | MX1,MX2 |
| | RSAD2 |
| | GBP2 |
| | CMPK2 |
| | OAS1, OASL |

ADCCR1 Cells Fail to Activate or Bind NK Cells:

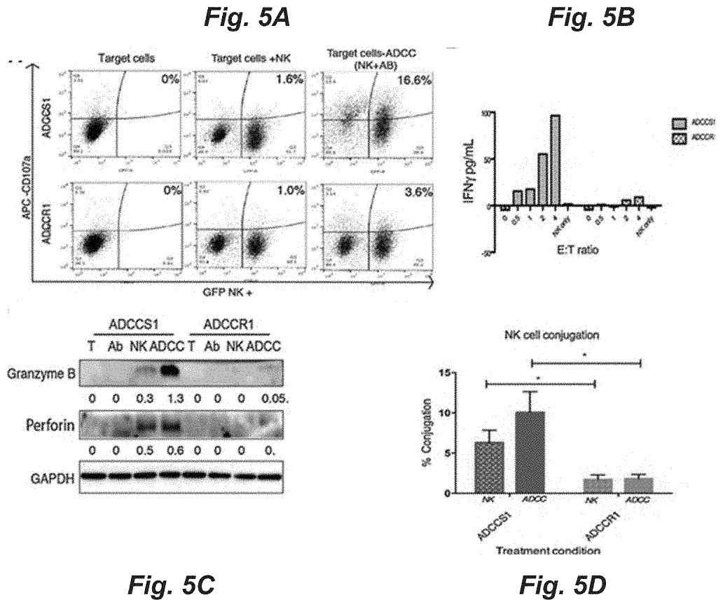

It was examined whether the resistance to ADCC-mediated lysis in ADCCR1 cells was due to an intrinsic mechanism (resistance to perforin/granzyme or blocking apoptosis) or to defective cell-cell conjugation. To assess NK activity, expression of CD107a, a marker of NK degranulation and activation, was quantified in the NK92-CD16V cells 2 hours after exposure to target cells in the absence or presence of cetuximab (FIG. 5A). CD107a was significantly increased in ADCCS1-exposed NK cells compared with ADCCR1-exposed NK cells (t test, P<0.0001). IFNγ secreted in the media post-ADCC was also measured (FIG. 5B). A correlation between the number of NK cells added and the amount of IFNγ released when using ADCCS1 cells was observed (P=0.001, R2=0.982), whereas no IFNγ was released at any effector-to-target ratio with ADCCR1 cells. Taken together, these results indicated that ADCCR1 cells failed to activate NK cells even in ADCC conditions. This was further verified by the absence of perforin and granzyme B released upon exposure of ADCCR1 cells to NK cells for 1 hour in the absence (NK lane) or presence of cetuximab (1 μg/mL; ADCC lane) by Western blot (FIG. 5C). In contrast, exposure of ADCCS1 cells to NK cells resulted in detectable levels of granzyme B and perforin by Western blot, even when exposed to NK cells alone. To further investigate ineffective NK cell activation by ADCCR1 cells, it was examined whether effector-target cell conjugation was occurring after treatment with NK cells in the presence or absence of cetuximab (1 μg/mL) for 4 hours. FIG. 5D shows the percentage of NK cells conjugated to target was measured by a multiwell conjugation assay as described in Materials and Methods. ADCCS1 (blue) and ADCCR1 (red) were incubated with NK92-CD16V cells at a 1:1 E:T ratio in the absence (NK, checkered bar) or in the presence of cetuximab (1 μg/mL; ADCC, solid bar) for 2 hours. *, P<0.05 by two-tailed t test. ADCCS1 cells conjugated to NK92-CD16V cells effectively in the presence and absence of cetuximab, whereas ADCCR1 cells' ability to conjugate was significantly less in both conditions (FIG. 5D). Therefore, ADCCR1 cells failed to activate NK cells and resisted ADCC-mediated lysis by avoiding NK cell conjugation.

ADCCR1 Cells Exhibit Reduced Expression of Multiple Cell-Surface Proteins:

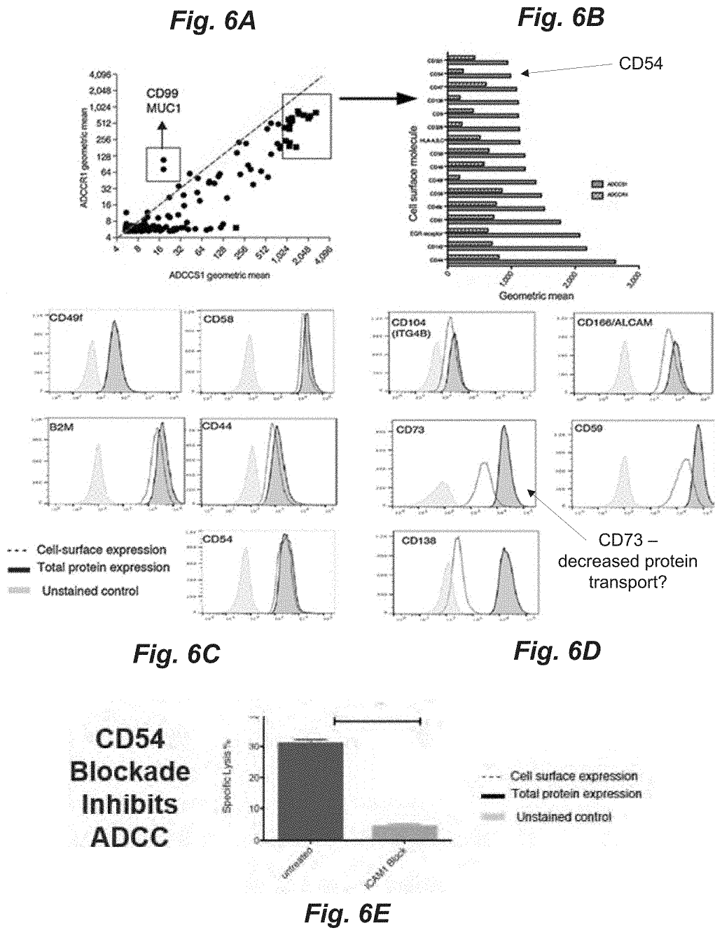

A BD Lyoplate cell-surface molecule screen was conducted to better understand the differences in conjugation of NK92-CD16V cells to ADCCS1 and ADCCR1 cells. FIGS. 6A-6D depict cell-surface screens of ADCCS1 and ADCCR1 cells. FIG. 6A, Dot plot comparing geometric means of ADCCS1 and ADCCR1 cell-surface molecule expression measured by the BD Lyoplate assay as described in Materials and Methods. Molecules with highest differential cell-surface expression in ADCCS1 cells are shown in the box. BD Lyoplate screen geometric means of ADCCS1 and ADCCR1 are shown in Table 1. FIG. 6B, Geometric means of molecules with the highest differential expression (box in A) in ADCCS1 (blue) compared with ADCCR1 (red) cells. FIG. 6C, Representative histograms of selected cell-surface molecules with reduced cell-surface expression on ADCCR1 cells based on lower protein expression. Light gray histograms, negative control; dark gray histograms, expression of total protein in permeabilized ADCCR1 cells; open histograms, cell-surface expression of indicated molecule in ADCCR1 cells. FIG. 6D, Representative histograms of selected cell-surface molecules with reduced cell-surface expression based on reduced transport to cell surface. Light gray histograms, negative control; dark gray histograms, expression of total protein in permeabilized ADCCR1 cells; open histograms, cell-surface expression of the indicated molecule in ADCCR1 cells. FIG. 6E presents the interesting finding that blockade of CD54 inhibits ADCC.

Many ADCCR1 cell-surface molecules were reduced compared with ADCCS1 cells, including cell adhesion molecules that play a role in the immune response, such as CD54 (ICAM-1), CD81 (TAPA-1), CD59, CD58, CD9, and HLA-A, —B, and -C (FIGS. 6A and 6B).

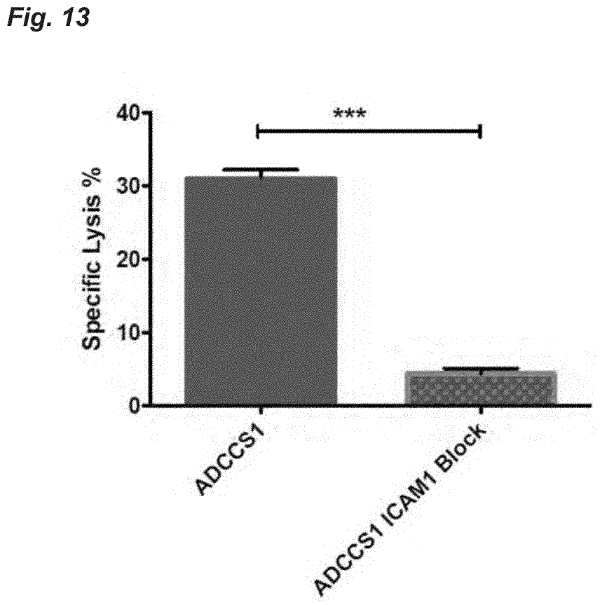

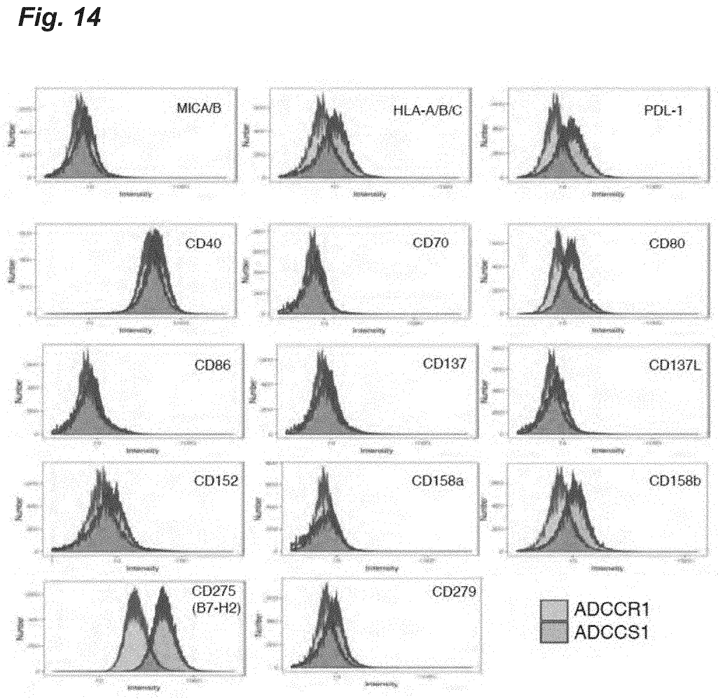

ICAM-1, a known LFA-1 ligand, was significantly downregulated in ADCCR1 cells, and LFA-1/ICAM-1 interactions are essential for NK cell activation. FIG. 13 shows specific lysis of untreated ADCCS1 (solid bar) and ADCCS1 treated with 10 μg/ml of ICAM-1 blocking antibody (checkered bar) as measured by cetuximab-mediated ADCC assay using NK92-CD16V effector cells. ***p<0.001 by two tailed t-test. Blocking LFA-1/ICAM-1 interactions significantly reduced ADCC sensitivity in ADCCS1 cells (FIG. 13). CD81 and CD9 are tetraspanin proteins that play roles in adhesion and formation of the immune synapse. Tetraspanins are known to associate at the immune synapse with receptors and integrins, including ICAM-1 and LFA-1. FIG. 14 shows immune checkpoint cell surface expression in ADCCS1 and ADCCR1 cells. Red histograms=ADCCR1; blue histograms=ADCCS1. Assay performed using BD-Lyoplate analysis as described in Materials and Methods. Results reflect changes as compared with negative control antibodies for each tested immune checkpoint. No upregulation of known immune checkpoints, including PD-L1, in ADCCR1 cells was observed (FIG. 14).

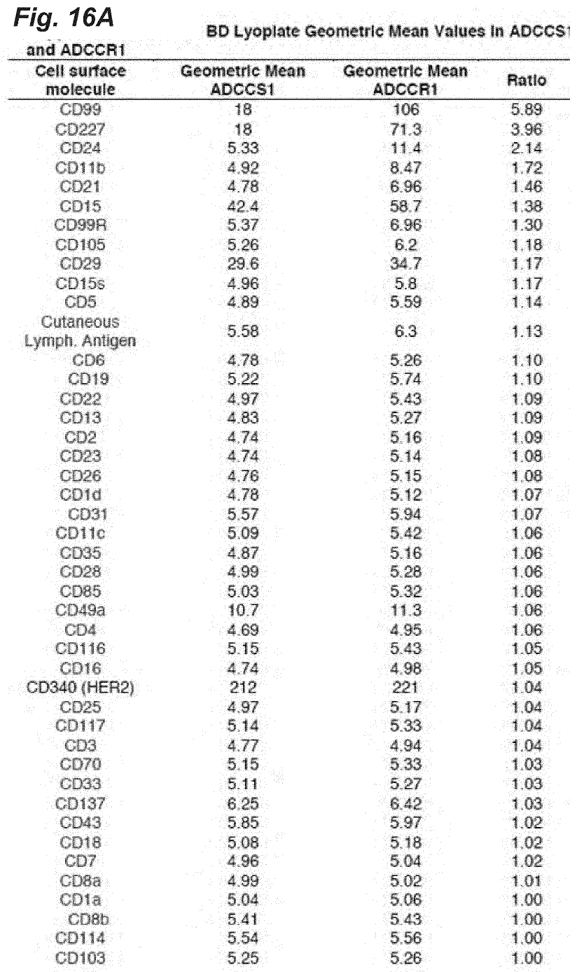

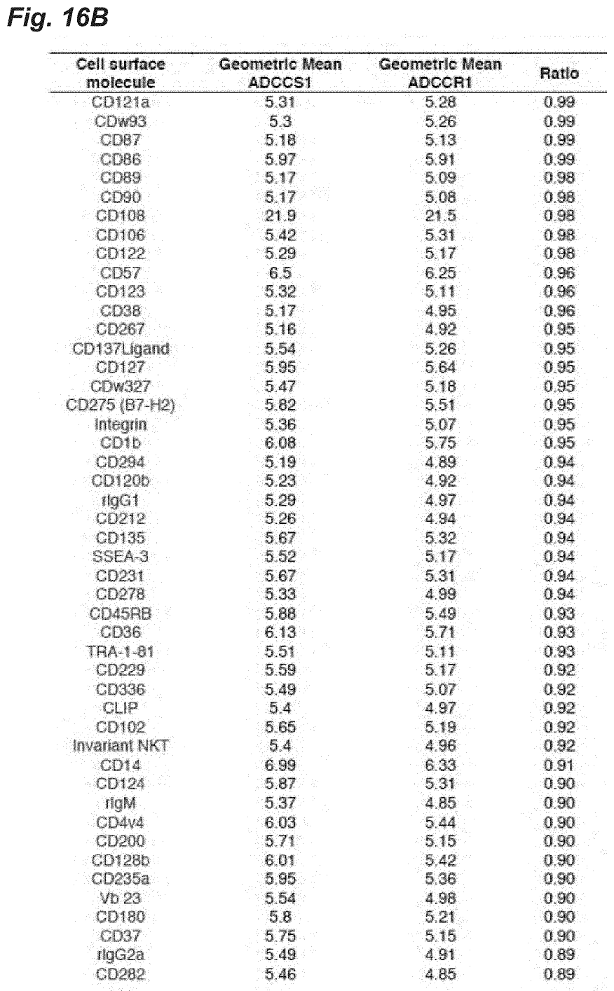

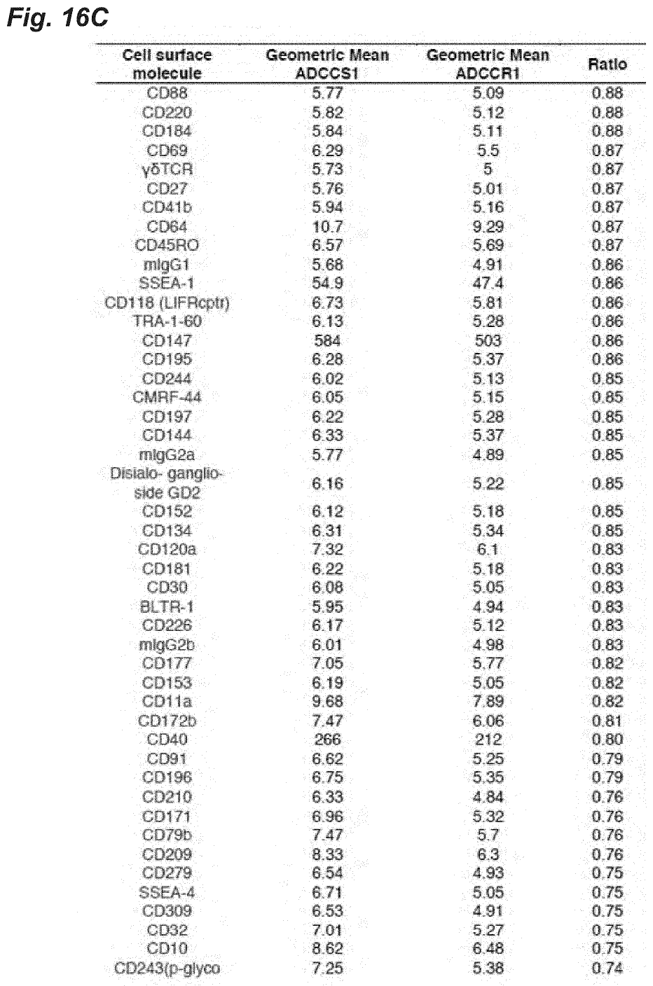

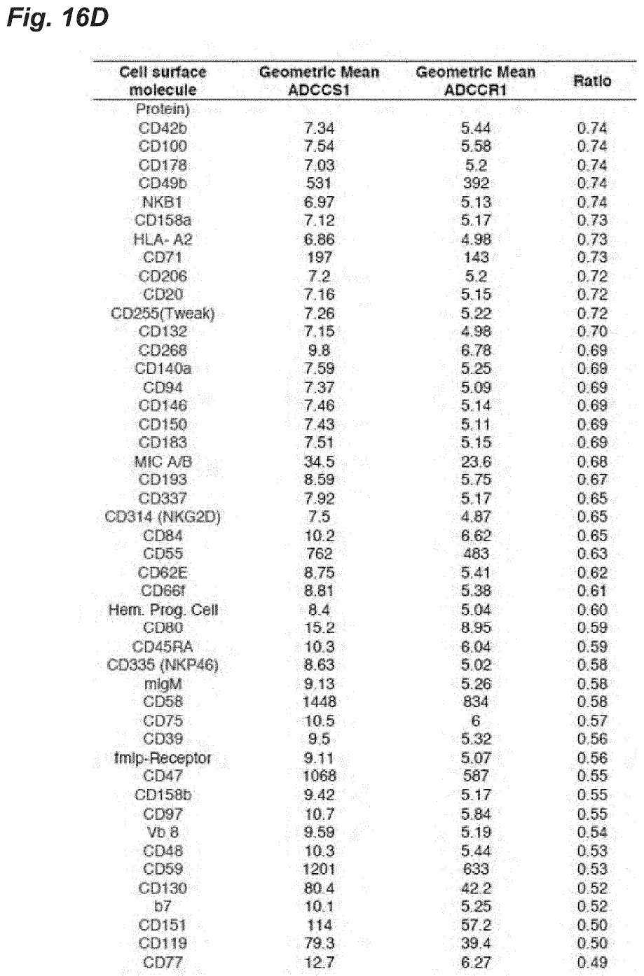

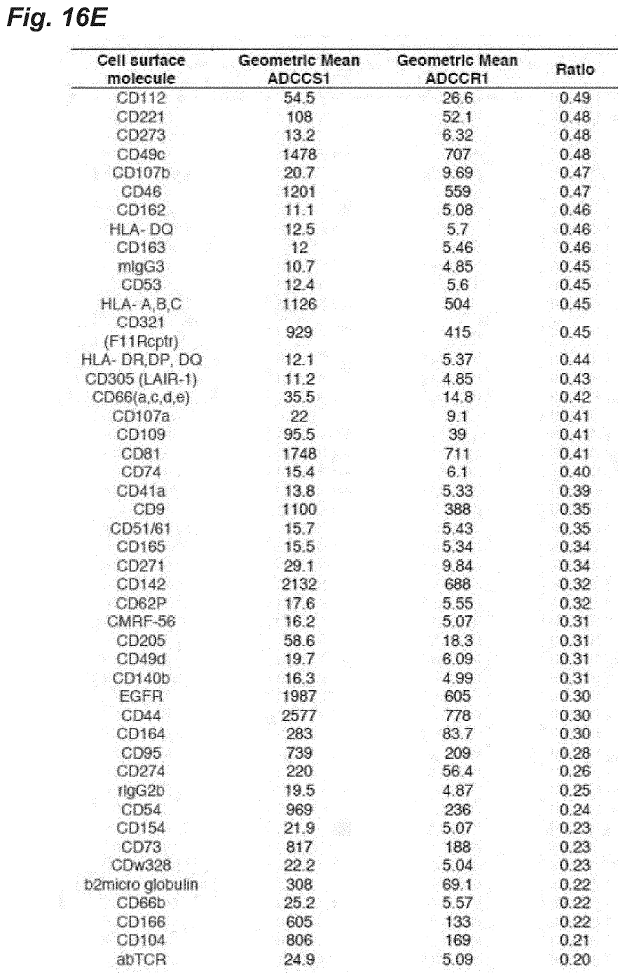

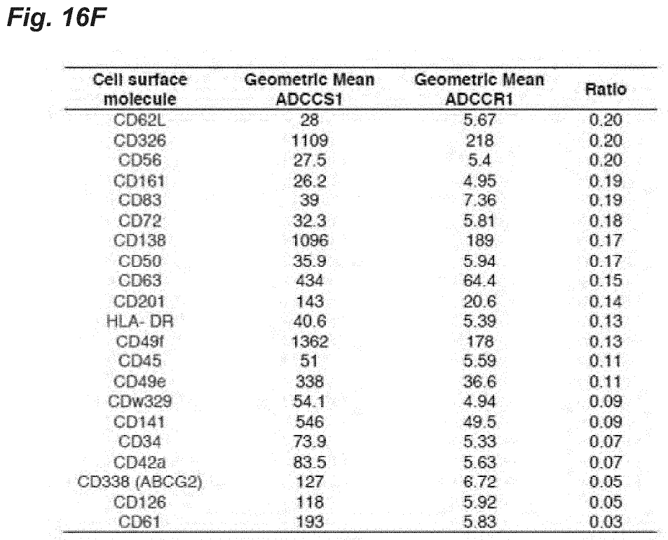

It was found that the reduced presence of select molecules on the cell surface did not necessarily correspond to a reduction in mRNA expression in ADCCR1 cells, with the exception of EGFR. Although some adhesion molecules with reduced cell-surface expression had concomitant reductions in protein expression, several molecules found to be downregulated in ADCCR1 cells on the cell surface did not have reduced protein expression, suggesting a failure of transport to the cell surface. Total BD Lyoplate geometric mean values of ADCCS1 and ADCCR1 are given in FIGS. 16A-16F.

Rederivation of ADCC Resistance:

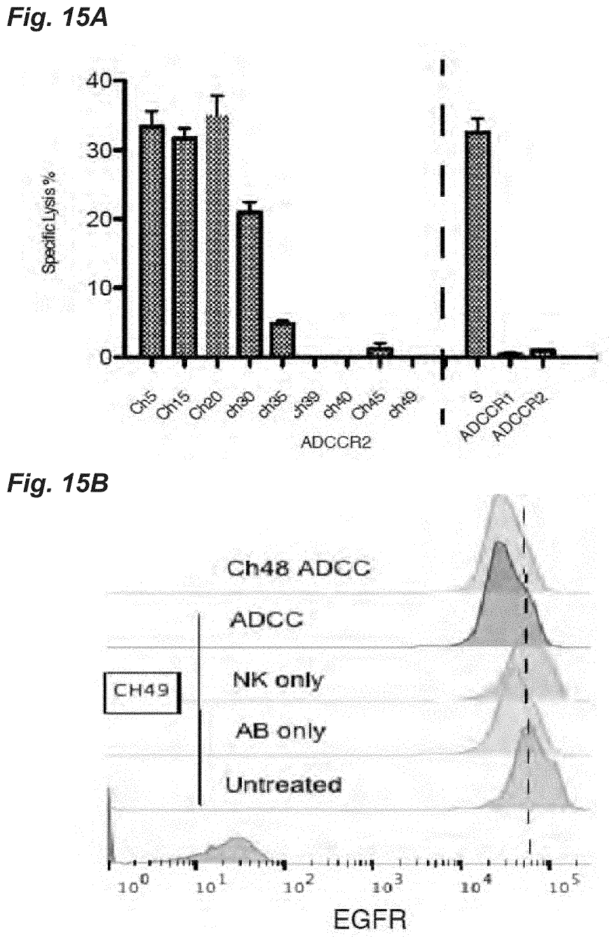

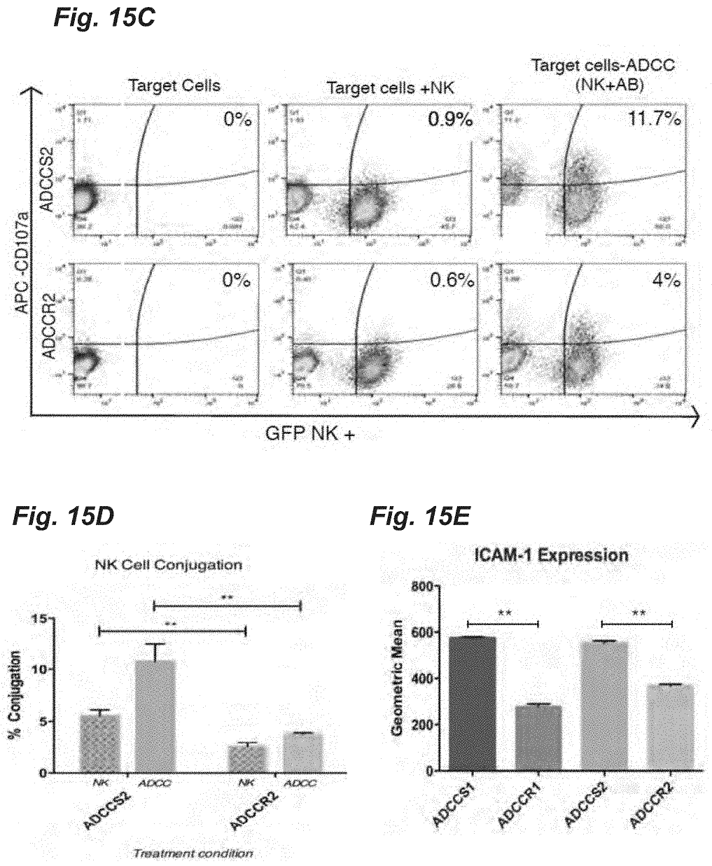

To shed light on the sequence of events as ADCC resistance develops, ADCC resistance was rederived from parental A431 cells by monitoring specific lysis under ADCC conditions, cell-surface EGFR expression, proliferation, and cellular morphology. A second ADCC resistant cell line was characterized with the results shown in FIGS. 15A-15E.

FIG. 15A shows specific lysis of A431 cells as measured by ADCC assay as a function of consecutive ADCC challenges as described in Materials and Methods during derivation of ADCCR2. Ch denotes the challenge number. Panel after dotted line compares specific lysis in control A431 (S), ADCCR1, and ADCCR2 at Ch49. FIG. 15B shows flow cytometry analysis of EGFR cell surface expression in ADCCR2 at challenge 48 and cells from the four challenge treatment conditions (untreated control, Ab treatment only, NK treatment only, and ADCC conditions) at challenge 49. FIG. 15C shows NK activation measured by flow cytometry analysis using CD107α (APC) and GFP+NK92-CD16V cells as described in Materials and Methods. ADCCS2 (top row) and ADCCR2 cells (bottom row) were incubated with NK92-CD16V cells in the absence (middle panels) or in the presence of 1 μg/ml of cetuximab (right panel) for 2 hours. FIG. 15D shows the percentage of NK cells conjugated to target was measured by multiwell conjugation assay as described in Materials and Methods. ADCCS2 (light blue) and ADCCR2 (orange) were incubated with NK92-CD16V cells in the absence (NK, checkered bar) or in the presence of 1 μg/ml of cetuximab (ADCC, solid bar) for 2 hours. ** p<0.01 by two tailed t-test. FIG. 15E shows the geometric means of ICAM-1 expression as measured by flow cytometric analysis in ADCCS1 (blue), ADCCR1 (red), ADCCS2 (light blue), and ADCCR2 (orange). ** p<0.01 by two tailed t-test.

Changes in morphology toward the appearance of ADCCR1 cells were first observed at challenge 27, whereas no significant changes in EGFR cell-surface expression or specific lysis was seen (FIG. 15A). ADCC resistance was first observed at challenge 39, with accompanying changes in cell proliferation, morphology, significantly reduced specific lysis, but no significant changes in cell-surface EGFR expression, as was seen in ADCCR1 cells. Despite an additional 10 ADCC challenges to these ADCC-resistant cells (ADCCR2), EGFR cell-surface expression was reduced in ADCCR2 by only 45% as compared with the 70% in ADCCR1 (FIG. 15B). Hence, the ADCC-resistance phenotype in these cells was unrelated to changes in cell-surface EGFR expression. Even without a significant loss of antibody target, the ADCCR2 line failed to activate NK cells (FIG. 15C) and displayed reduced NK cell conjugation (FIG. 15D). ADCCR2 cells also exhibited reduced ICAM-1 expression similar to ADCCR1 cells (FIG. 15E), suggesting that ADCC resistance in both lines can be attributed to evasion of NK cell binding.

### Example 3

### Deriving Resistance to ADCC

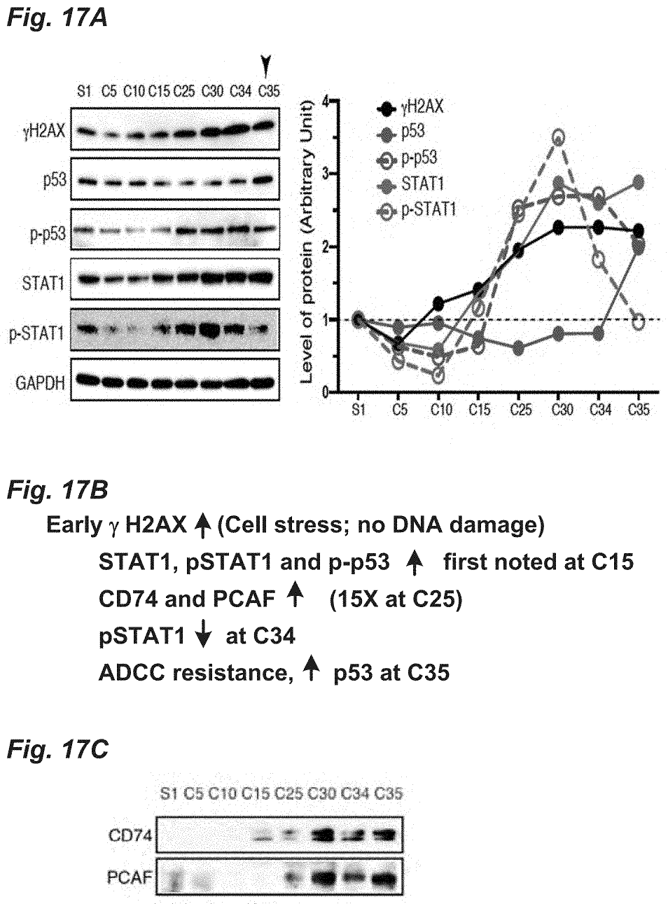

Molecular Sequence of Events Leading to Ability of Tumor Cells to Evade Conjugation with Cytotoxic Apparatus of the Immune System: In another embodiment, the inventor set out to determine the molecular changes occurring in tumor cells as they become ADCC resistant. In FIG. 17A, results of evaluation of the expression of markers γH2AX, p53, p-p53, STAT1, p-STAT1 compared with housekeeping marker GAPDH is shown with the number of successive ADCC challenges. H2AX is a variant of the H2A protein family, which is a component of the histone octamer in nucleosomes. It has been reported to be phosphorylated by kinases such as ataxia telangiectasia mutated (ATM) and ATM-Rad3-related (ATR) in the PI3K pathway. The newly phosphorylated protein, gamma-H2AX (γH2AX), is the first step in recruiting and localizing DNA repair proteins. P53 is a tumor suppressor that regulates cell division by keeping cells from growing and dividing proliferating too fast or in an uncontrolled way. P53 is phosphorylated by posttranslational modification of p53 to form p-p53, which has been proposed to be an important mechanism by which p53 stabilization and function are regulated. Signal transducer and activator of transcription 1 (STAT1) is a transcription factor encoded by the STAT1 gene in humans and is a member of the STAT protein family. STAT1 is involved in upregulating genes due to a signal including by the type I, type II, or type III interferons, Epidermal Growth Factor (EGF), Platelet Derived Growth Factor (PDGF) and Interleukin 6 (IL-6). Activated STAT1 occurs via phosphorylation (p-STAT1) by receptor associated kinases resulting in dimerization and translocation to nucleus to work as a transcription factor. Cluster of Differentiation 74 (CD74) is the HLA class II histocompatibility antigen gamma chain, also known as HLA-DR antigen-associated invariant chain. Milatuzumab (or hLL1) is an anti-CD74 humanized monoclonal antibody being studied for the treatment of multiple myeloma, non-Hodgkin's lymphoma and chronic lymphocytic leukemia. P300/CBP-associated factor (PCAF), also known as K(lysine) acetyltransferase 2B (KAT2B), is a human gene and transcriptional coactivator associated with p53. In FIG. 17C, results of evaluation of the expression of markers CD74 and PCAF is shown with the number of successive ADCC challenges, both genes being highly expressed by C30. As shown in FIG. 17B, early γH2AX is upregulated but levels off indicating cell stress without DNA damage, STAT1, pSTAT1 and p-p53 are upregulated first noted at C15, CD74 and PCAF are upregulated up to 15× by C25. Interestingly pSTAT1 decreases at C34 coincident with ADCC resistance and increased p53 at C35.

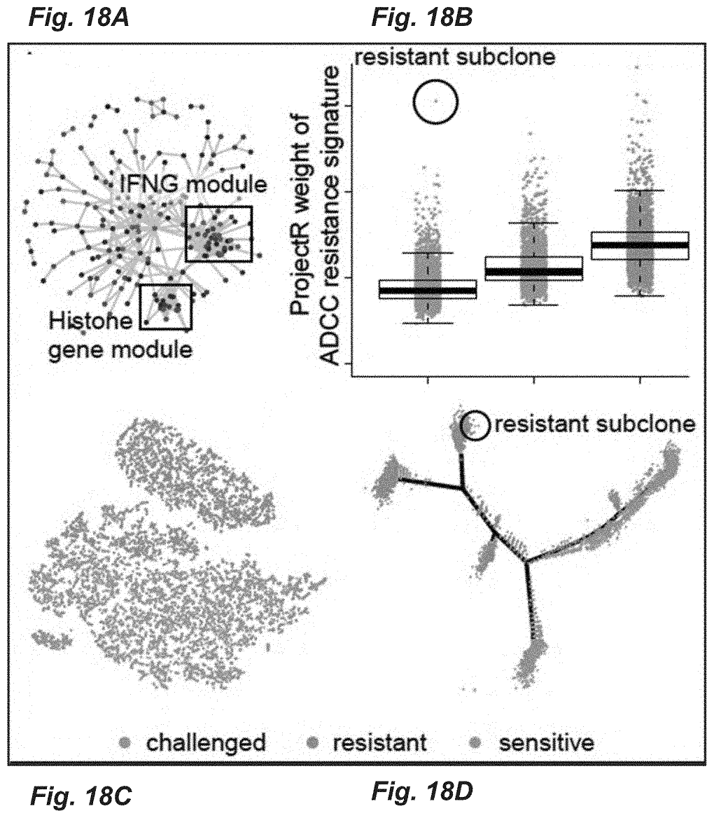

FIG. 18A-18D show expansion of pre-existing resistant clones. 10× scRNAseq analysis was conducted and identified a ADCCS1 cell with an ADCCR1 resistance signature. FIG. 18A shows the results of CoGAPS analysis of bulk gene expression profiling. FIG. 18B applies ProjectR to transfer the resistance signature learned in bulk data. FIG. 18C shows UMAP analysis clusters suggesting incomplete penetrance of the resistant phenotype, while FIG. 18D reveals that similar trends are observed in pseudotime analysis.

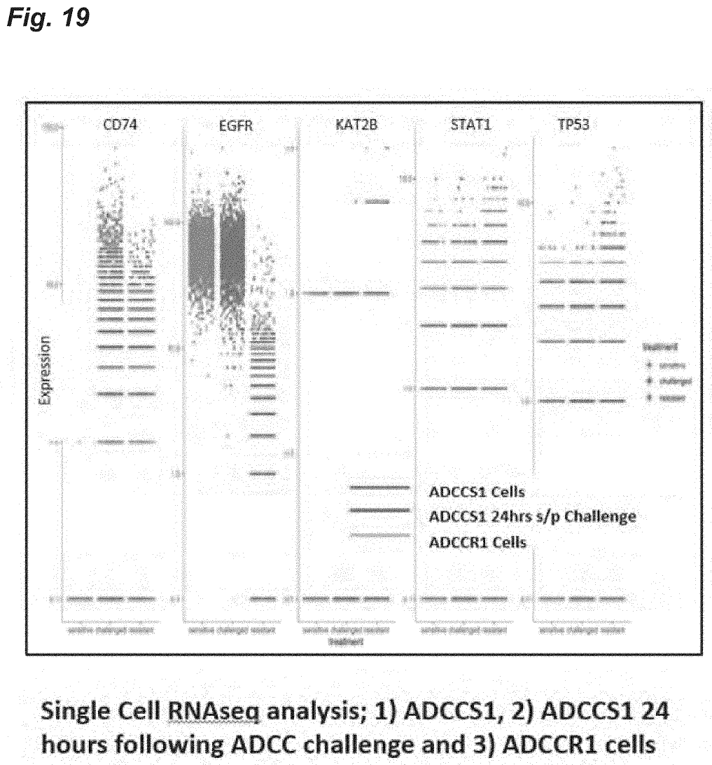

FIG. 19 depicts the results of single cell RNAseq analysis of 1) ADCCS1, 2) ADCCS1 24 hours following ADCC challenge and 3) ADCCR1 cells for CD74, EGFR, KAT2B (PCAF), STAT1, and p53.

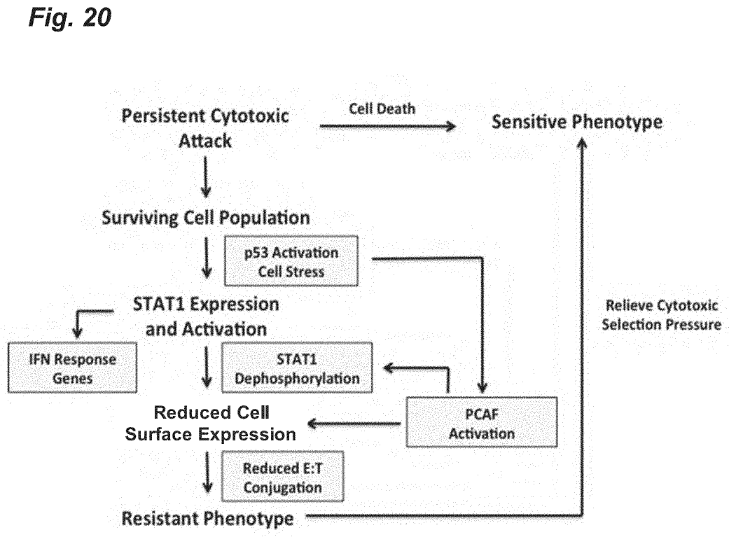

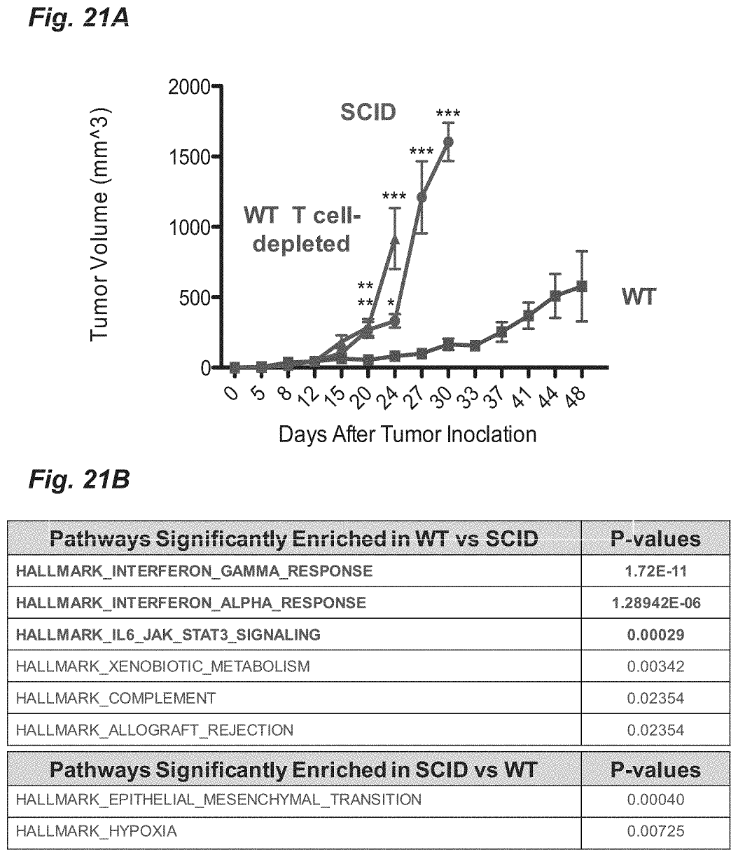

FIG. 20 presents a pathway of resistance development indicating that reduced cell surface expression of certain molecules results in ADCC resistance leading to the question of how the malignant epithelial cell is able to respond to the immune selection pressure.

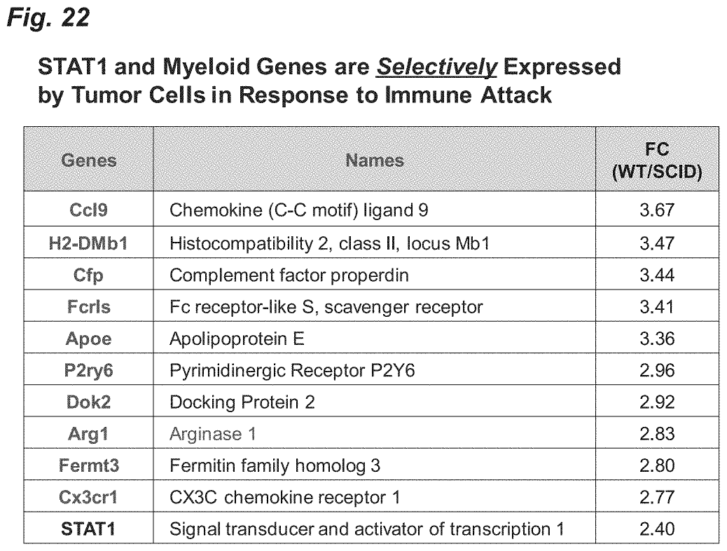

FIG. 21A shows the role of T-cell immunity in a pancreatic cancer cell model where MT3-2D pancreatic cancer cells are introduced into immunocompetent (WT) C57Bl/6 mice versus SCID C57/BL mice and T-cell depleted WT mice. As can be seen, the tumor is very aggressive in SCID and T-cell depleted mice but also continues to grow in the WT mice. RNAseq analysis revealed significantly different gene expression in WT vs SCID malignant epithelial cells. FIG. 21B shows the pathways significantly enriched in WT vs SCID and the reciprocal. As shown in FIG. 22, it was determined that STAT1 and myeloid genes were selectively expressed by tumor cells in response to immune attack.

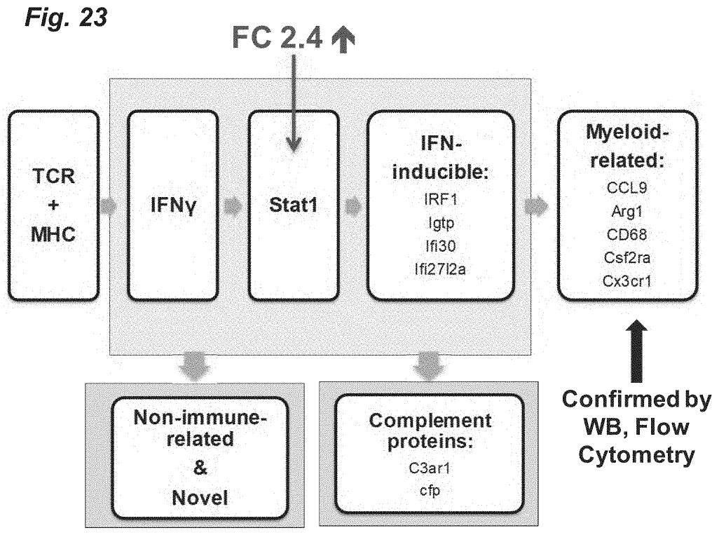

FIG. 23 presents a preliminary model on the RNAseq data.

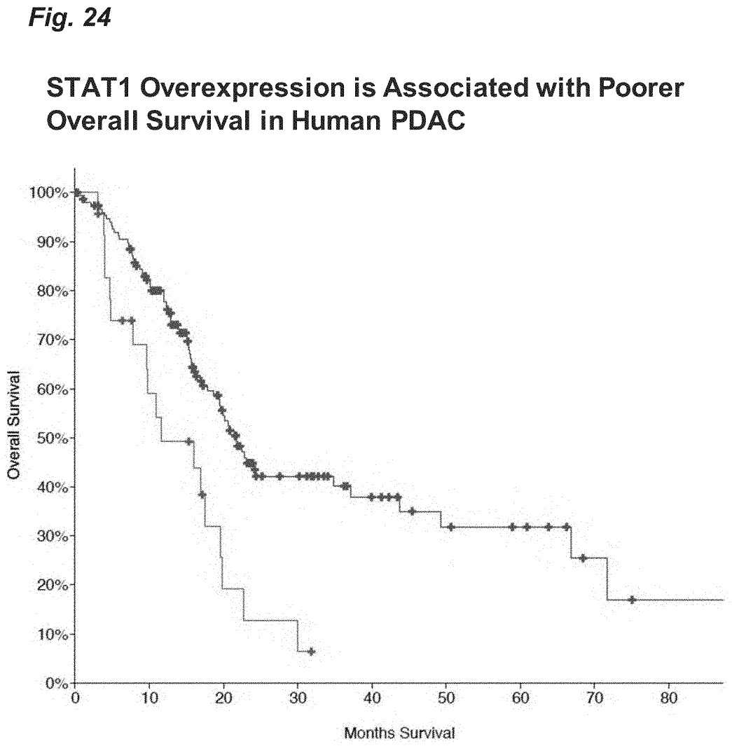

FIG. 24 presents data the STAT1 overexpression is associated with poorer overall survival in human PDAC.

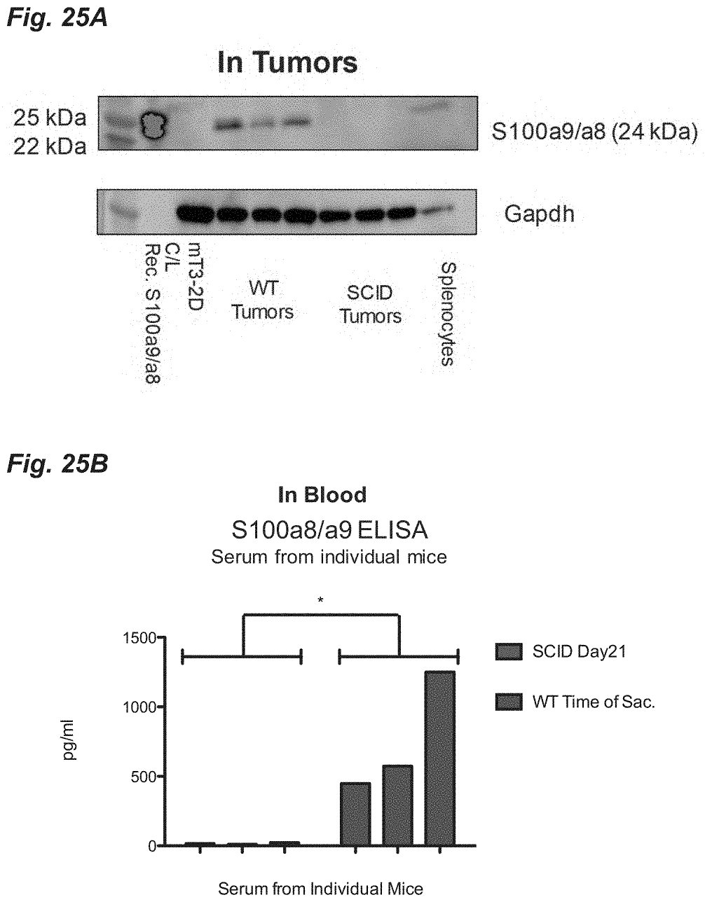

FIGS. 25A and 25B present data showing that S100a8/a9 is selectively increased in WT tumors by proteomic analysis, is not expressed by tumor cells, but is clearly induced by T cell immunity. S100 calcium-binding protein A9 (S100A9) also known as migration inhibitory factor-related protein 14 (MRP14) or calgranulin B and is a protein that in humans is encoded by the S100A9 gene. The proteins S100A8 (MRP-8) and S100A9 form a heterodimer called calprotectin. MRP14 complexes with MRP-8 and together MRP8 and MRP14 regulate myeloid cell function including by binding to Toll-like receptor 4. MRP-8/14 broadly regulates vascular inflammation and contributes to the biological response to vascular injury by promoting leukocyte recruitment. This molecule is controlled in part by STAT1, binds to RAGE and activates MDSC.

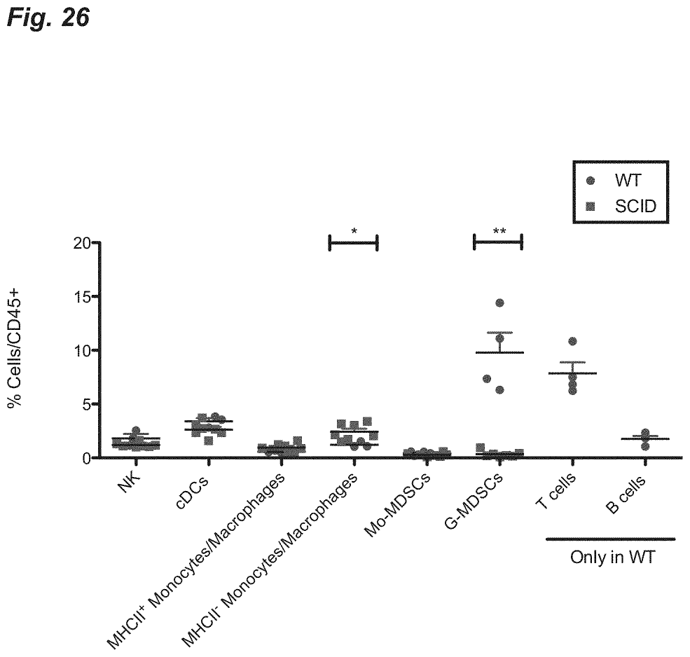

FIG. 26 presents data showing that neutrophilic myeloid derived suppressor cells (G-MDSC) selectively accumulate in WT mouse tumors. Thus in one model, the data suggests that tumors are effective in a myriad of ways including: they overwhelm the immune response by out-proliferation; they hide by decreased expression of target antigens, WIC Class I/II molecules and cell adhesion proteins; they subvert the immune system with immunosuppressive chemokines, cytokines (e.g., M2, Th2 phenotypes) possibly under control of STAT1; they shield themselves by excluding infiltration by tumor antigen-reactive T cells; and they defend themselves by deactivating tumor-targeting T cells that attack tumor cells.

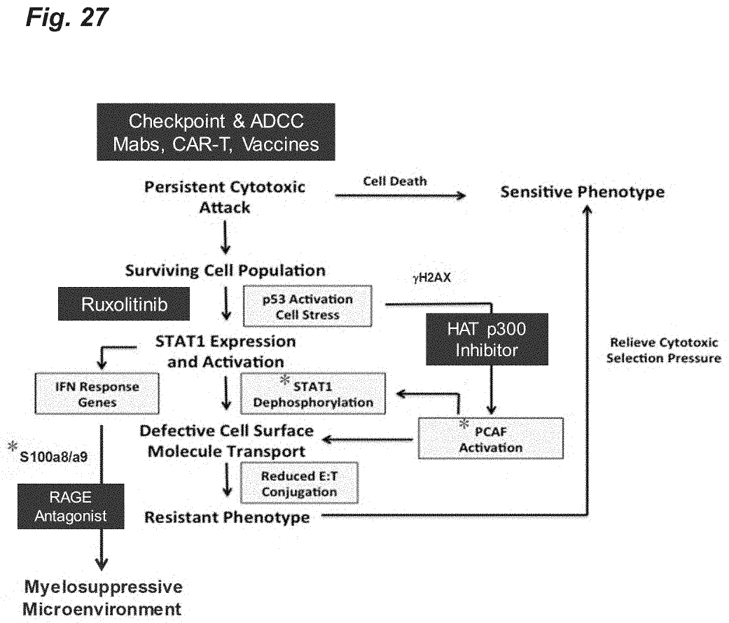

FIG. 27 presents the new pathways uncovered leading to immune and immunotherapy resistance and defines new targets for combination therapy. In one prong in the attack on immunotherapy resistance, STAT1 expression is targeted. As shown in FIG. 27, in one embodiment STAT 1 is targeted through treatment with Ruxolitinib. Ruxolitinib is a janus kinase inhibitor (JAK inhibitor) with selectivity for subtypes JAK1 and JAK2. JAK1 and JAK2 are membrane receptor-associated Janus kinases, which are known to participate in activation of STAT transcription factors following signaling by several ligands including interferon alpha (IFNα), interferon beta (IFNβ), interferon gamma (IFNγ), Epidermal Growth Factor (EGF), Platelet Derived Growth Factor (PDGF) or Interleukin 6 (IL-6) leading to modulation of gene expression of the interferon stimulated genes. By inhibiting STAT1, the resistant phenotype consequent to reduced expression of cell surface molecules required for effective docking of NK cells via tumor specific antibodies will be reduced.

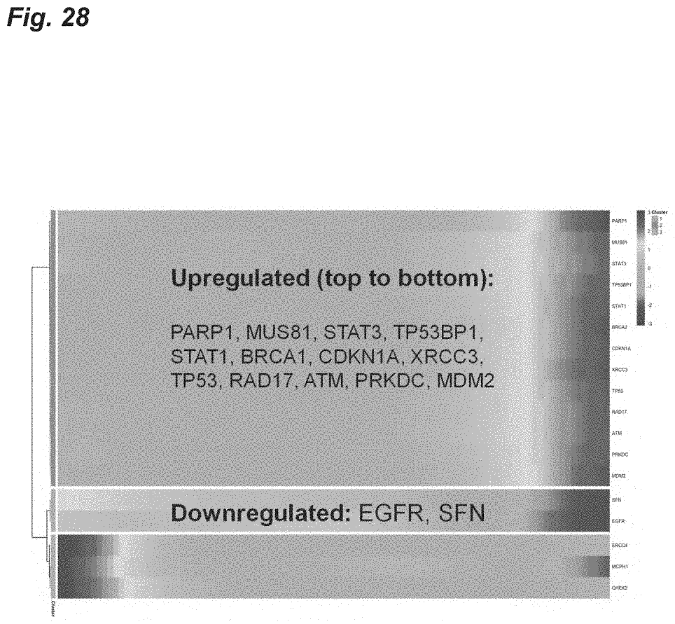

FIG. 28 presents the results of a single cell RNAseg analysis of PARP1, MUS81, STAT3, TP53BP1, STAT1, BRCA1, CDKN1A, XRCC3, TP53, RAD17, ATM, PRKDC, MDM2, EFGR, and SFN with activation signature in ADCC resistance. As shown, PARP1, MUS81, STAT3, TP53BP1, STAT1, BRCA1, CDKN1A, XRCC3, TP53, RAD17, ATM, PRKDC, MDM2, EFGR, and SFN are upregulated, while EGFR and SFN are downregulated.

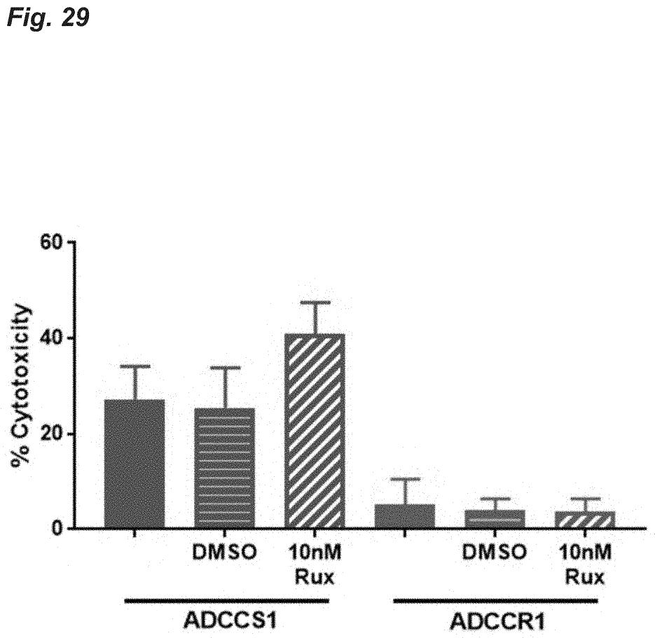

FIG. 29 presents the results of a cytotoxicity analysis of ADCCS1 and ADCCR1 after ruxolitinib exposure. As shown, ruxolitinib exposure increases the ADCC magnitude of cytotoxicity in ADCCS1 cells but not in ADCCR1 cells.

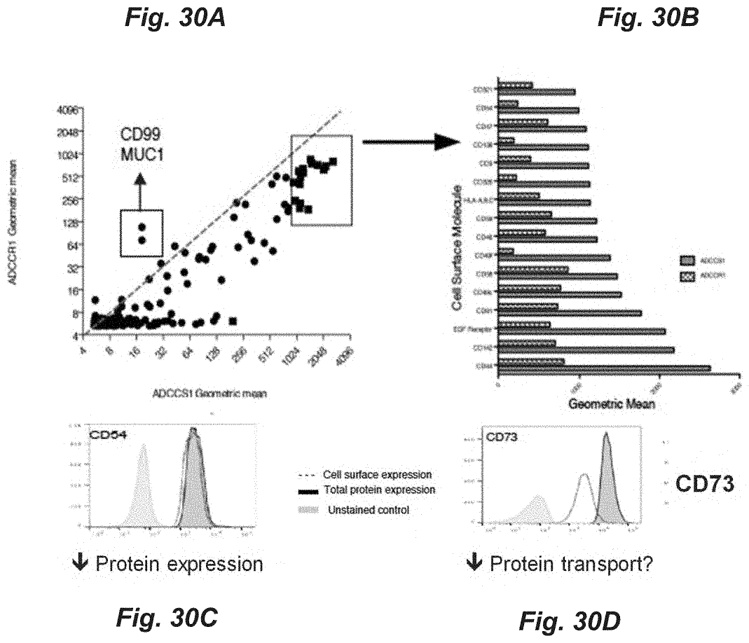

FIG. 30A-D present data showing that ADCC resistance is associated with reduced cell surface expression of multiple proteins. FIG. 30A shows the results of a BD lyoplate assay that demonstrates that CD99 and MUC1 surface expression increases ADCCS1 cell lines. FIG. 30B shows the expression of a number or surface molecules, including CD54 expression in ADCCS1 and ADCCR1 cells, which shows that CD54 surface expression is decreased in ADCCR1 cells. FIG. 30C shows CD54 protein expression, both at the cell surface and in total, as compared to an unstained control. FIG. 30D shows an analogous analysis of CD73 protein surface expression. However, it appears that gene expression, in general, of the above-analyzed proteins in the cell lines remains generally unchanged.

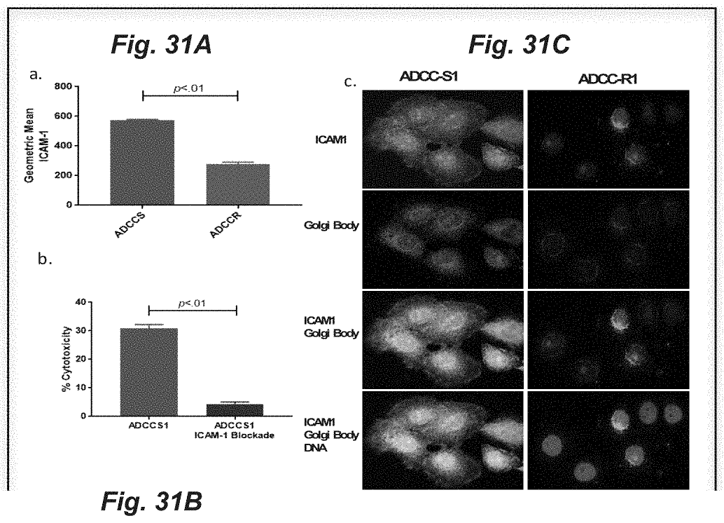

FIG. 31A-C present data showing that ADCC resistance is accompanied by the loss of CD54 associated with sequestration in Golgi complexes. FIG. 31A shows that CD54 expression in ADCC resistant cells is reduced compared to ADCC sensitive cells. FIG. 31B shows that a CD54 blockade reduces ADCC in ADCC sensitive cells. FIG. 31C shows that intracellular sequestration of CD54 is associated with Golgi complexes.

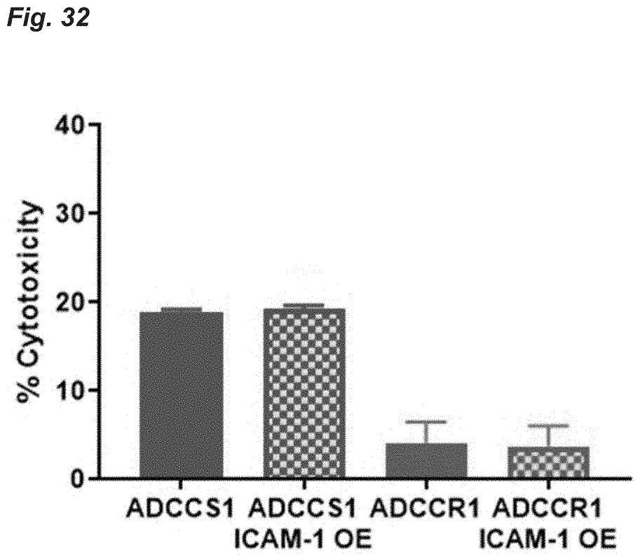

FIG. 32 presents the results of ICAM1 re-expression on ADCC sensitivity. As shown, there is no effect from ICAM-1 re-expression on ADCC sensitivity in either ADCCS1 or ADCCR1 cells.

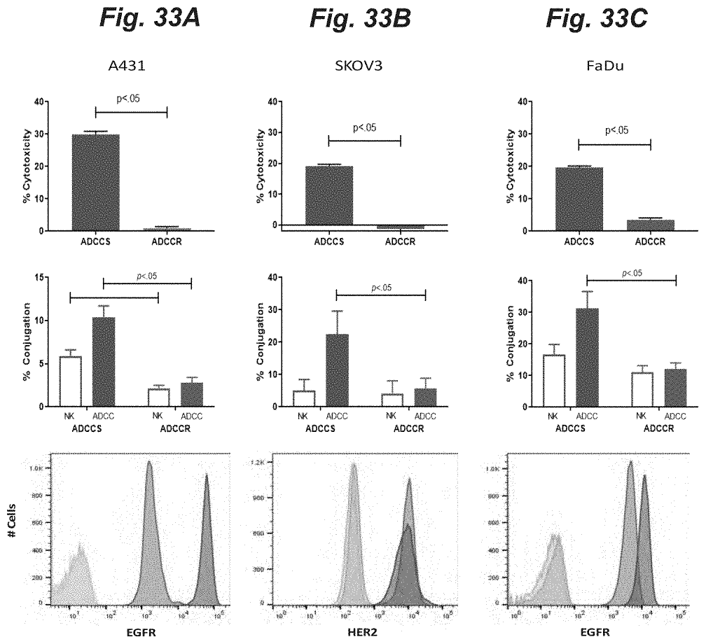

FIG. 33A-C present data showing the ADCC resistance mechanism reproduced in multiple cell lines. FIG. 33A shows the ADCC resistance mechanism in A431 cell lines. FIG. 33B shows the ADCC resistance mechanism in SKOV3 cell lines. FIG. 33C shows the ADCC resistance mechanism in FaDu cell lines.

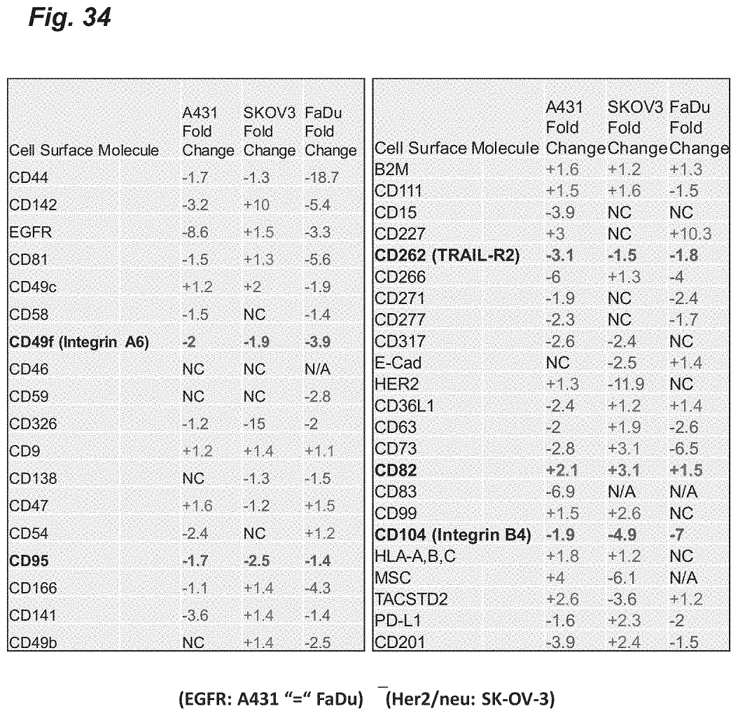

FIG. 34 shows the results of LEGENDscreen of surface molecules of ADCC resistant cells of A431, SKOV3, and FaDu cell lines. The results reveal divergent surface protein expression patterns in ADCC resistant cells. Notably, CD82 (tetraspanin-27 protein) is gained in ADCC resistant cells. CD262 (DR-5, TRAIL-R2 protein), CD95 (Fas receptor protein), CD104 (integrin β4 protein), and CD49f (integrin a6 protein) are lost in ADCC resistant cells.

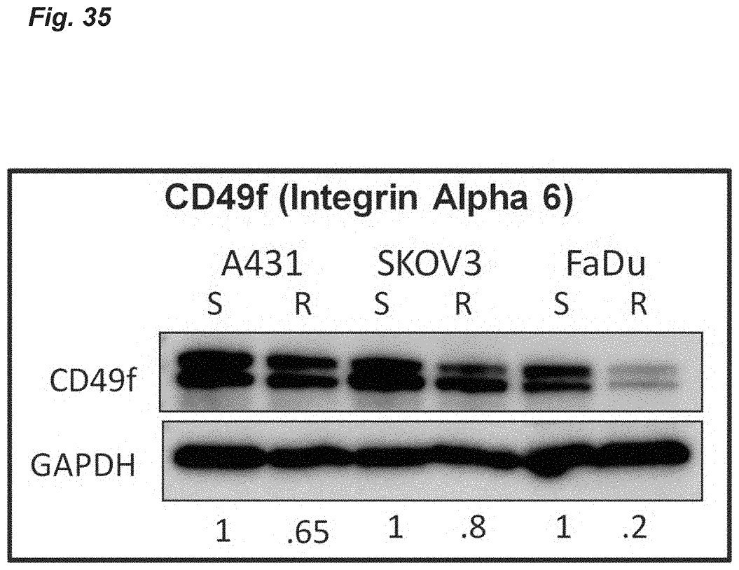

FIG. 35 shows the results of immunoblotting the expression of CD49f and GAPDH in ADCC sensitive and ADCC resistant cells of the A431, SKOV3, and FaDu cell lines. As shown, the FaDu ADCC resistant cells show reduced expression of CD49f.

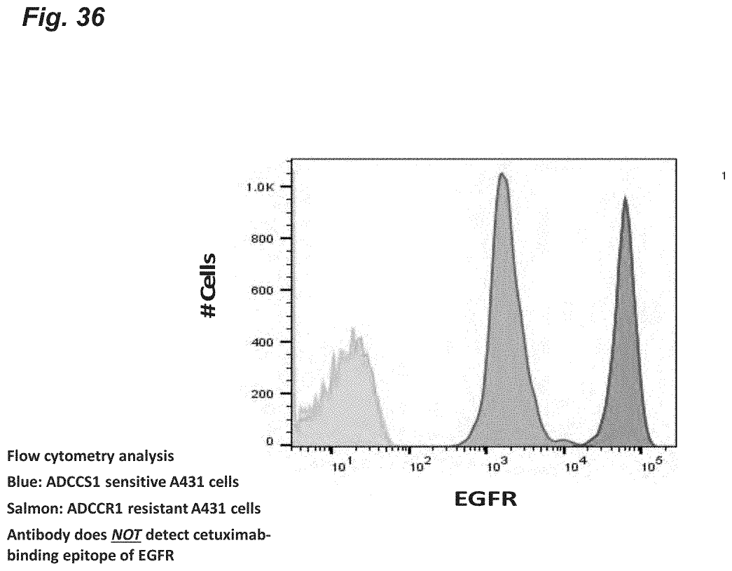

FIG. 36 shows the results of a flow cytometry analysis of ADCCS1 and ADCCR1 cells for expression of EGFR and the cetuximab-binding epitope of EGFR. The ADCCS1 cells are shown in blue, while the ADCCR1 cells are shown in salmon-color. As shown, the ADCCR1 cells express EGFR but are not bound by directly-labeled cetuximab. That is evidenced by the fact that the antibody does not detect the cetuximab-binding epitope of EGFR.

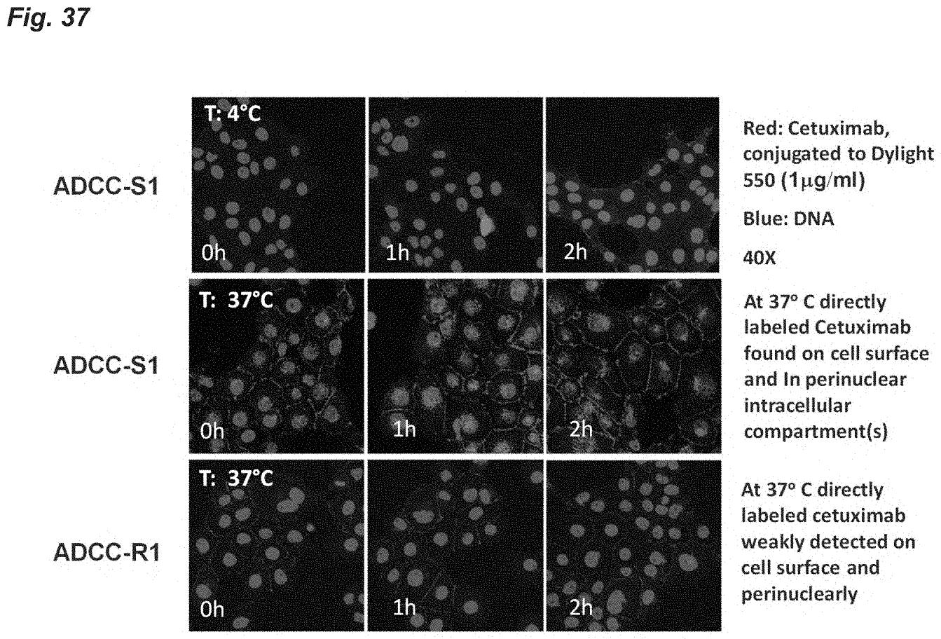

FIG. 37 shows the results of immunofluorescent imaging for cetuximab in ADCCS1 and ADCCR1 cells of the A431 cell line. As previously discussed, ADCCR1 cells express EGFR but are not bound by directly-labeled cetuximab. That conclusion is confirmed by the fact that directly labeled cetuximab is weakly detected on the cell surface and perinuclearly of ADCCR1 cells, while it is detected more strongly on cell surface and perinuclearly in ADCCS1 cells.

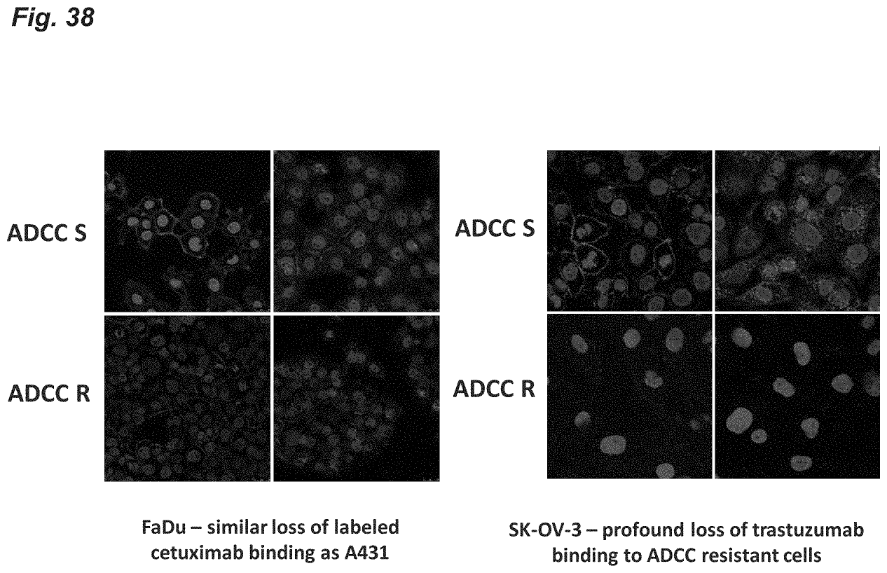

FIG. 38 shows the results of immunofluorescent imaging for cetuximab and trastuzumab in ADCC sensitive and ADCC resistant cells in the FaDu and SKOV3 cell lines. In ADCC resistant cells of the FaDu cell line, there is a similar loss of labeled cetuximab binding to that in A431 cell lines. In ADCC resistant cells of the SKOV3 cell line, there is a profound loss of trastuzumab binding.

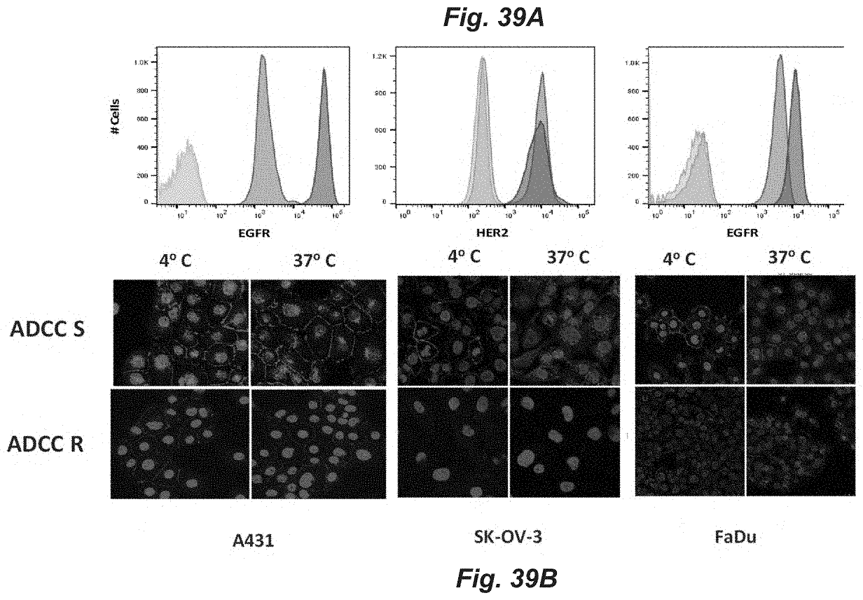

FIG. 39A-B shows the results of flow cytometric and immunofluorescence analysis, demonstrating that ADCC-resistant cells exhibit loss of binding of directly-labeled antibody to target antigens. FIG. 39A shows flow cytometry based binding of commonly used flow antibodies to target cells. Blue—Sensitive Cells; Salmon—Resistant Cells; Mauve—Negative Control. FIG. 39B shows directly-labeled antibodies binding to ADCC-resistant cells: Cetuximab (A431, FaDu) or trastuzumab (SK-OV-3), conjugated to Dylight 550 (1 mg/ml). Blue: DNA. 40X. As shown in those figures, none of the directly-labeled antibodies bind well to ADCC-resistant cells

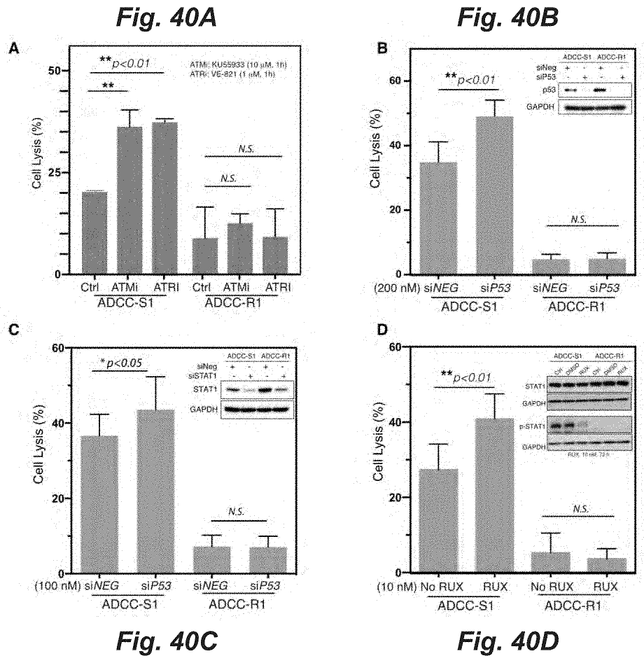

FIG. 40A-D show the effects of ATMi, ATM, siP53, and RUX (ruxolitinib) on cell lysis in ADCCS1 and ADCCR1 cells. FIG. 40A shows cell lysis rates for ATMi and ATM. FIG. 40B shows cell lysis rates for 200 nM siP53. FIG. 40C shows cell lysis rates for 100 nM siP53. FIG. 40D shows cell lysis rates for 10 nM ruxolitinib. In general, it is evident from the cell lysis percentages that ADCC resistant cells exhibit lower rates of cell lysis in response to ATMi, ATM, siP53, and RUX (ruxolitinib) compared to ADCC sensitive cells.

In another prong in the attack on immunotherapy resistance, a RAGE (Receptor for Advanced Glycation Endproducts) antagonist is employed. One such antagonist is the small molecule Azeliragon. A number of antibodies against RAGE are available although none are currently FDA approved. RAGE, also called AGER, is a 35 kilodalton transmembrane receptor of the immunoglobulin super family that has a role in as a pro-inflammatory gene activator, particularly in innate immunity. In certain mouse models of inflammation-associated skin, colon and liver carcinogenesis, activation of RAGE and/or NF-κB signaling result in strong upregulation of S100A8/A9 in keratinocytes, myeloid cells and tumor cells. The RAGE antagonist is employed to inhibit upregulation of S100A8 and S100A9 thus inhibiting development of a myelosuppressive microenvironment.

In yet another prong of the attack on immunotherapy resistance, a Histone Acetyltransferase (HAT) p300 inhibitor is employed to inhibit the reduced cell surface expression of molecules found here to contribute to development of an ADCC resistant phenotype. Histone acetyltransferase enzymes are also called lysine acetyltransferases (KATs) consequent to understanding of a great number of substrates for the enzymes. HAT p300 is also known as KAT3A. HATp300 and its paralog CREB-binding protein (CBP), now called KAT3B, have a myriad of defined histone and nonhistone substrates and are known to interact with hundreds of cellular binding partners. HATp300 is known to participate in regulation of NK-kB and p53 among others. The first p300 inhibitor was a Lys-coenzyme A conjugate, designed as a bisubstrate inhibitor. (Lau et al. HATs off: Selective Synthetic Inhibitors of the Histone Acetyltransferases p300 and PCAF Molecular Cell 5 (2000) 589-595. Lau also described another coenzyme A conjugate with a histone H3 peptide was shown to function as a selective PCAF inhibitor. Small molecule inhibitors of HAT p300/KAT3A are available including C646, a pyrazolone-furan, which is a highly selective against p300 and has been shown to decrease pro-inflammatory gene expression and NFκB activity and inhibit histone deacetylases.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A method of inhibiting acquisition of resistance to T cell mediated killing of tumor cells, the method comprising reducing the expression or activity of one or more cell adhesion molecules in a tumor cell.

2. The method of claim 1, wherein the cell adhesion molecules are one or more of STAT1, acetyl transferase p300, and S100A8/A9.

3. The method of claim 2, wherein STAT1 expression or activity is reduced using a therapeutically effective amount of ruxolitinib.

4. A method of identifying inhibitors of development of an antibody-dependent cellular cytotoxicity (ADCC) resistant phenotype, the method comprising adding potential inhibitors to a model of ADCC resistance wherein the model was developed by repeated ADCC challenge to tumor cells in culture.

5. A method of identifying agents able to reverse an antibody-dependent cellular cytotoxicity (ADCC) resistant phenotype, the method comprising adding potential agents to a model of ADCC resistance wherein the model was developed by repeated ADCC challenge to tumor cells in culture.
